(19) United States
(12) Patent Application Publication
(10) Pub. No.: US 2011/0130390 A1
(43) Pub. Date: Jun. 2, 2011

(54) Use of COX-2 Inhibitors for the Treatment of Schizophrenia, Delusional Disorders, Affective Disorders, Autism or Tic Disorders

(76) Inventor: Norbert Müller, Munich (DE)

(21) Appl. No.: 12/983,585

(22) Filed: Jan. 3, 2011

Related U.S. Application Data
(63) Continuation-in-part of application No. 10/480,600, filed on Feb. 5, 2004, now abandoned.
(60) Provisional application No. 60/364,904, filed on Mar. 14, 2002.

(30) Foreign Application Priority Data
Jun. 19, 2001 (DE) 101 29 320.8

(51) Int. Cl. A61K 31/5513; A61K 31/415; A61K 31/42; A61K 31/341; A61K 31/444; A61K 31/421; A61K 31/50; A61P 25/00

(52) U.S. Cl. 514/221; 514/406; 514/378; 514/473; 514/334; 514/374; 514/247

(57) ABSTRACT

The invention concerns the use of a COX-2 inhibitor for the treatment of psychiatric disorders such as schizophrenia, delusional disorders, affective disorders, autism or tic disorders, in particular chronic schizophrenic psychoses and schizoaffective psychoses, temporary acute psychotic disorders, depressive episodes, recurring depressive episodes, manic episodes and bipolar affective disorders. Moreover, the invention is concerned with the use of a COX-2 inhibitor, in particular celecoxib, in combination with a neuroleptic drug, in particular risperidone, or an antidepressant, for the treatment of psychiatric disorders such as schizophrenia, delusional disorders, affective disorders, autism or tic disorders.

This application is a continuation-in-part of U.S. patent application Ser. No. 10/480,600, filed Feb. 5, 2004, which claims priority under to PCT International Application No. PCT/EP02/06013, filed May 31, 2002, which claims priority to Provisional Application No. 60/364,904, filed Mar. 14, 2002, and claims priority under 35 U.S.C. 119 to German Patent Application No. 101 29 320.8, filed Jun. 19, 2001, the entire disclosures of which is herein expressly incorporated by reference.

### SUMMARY OF THE INVENTION

The invention concerns the use of a COX-2 (cyclooxygenase-2) inhibitor for the treatment of psychiatric disorders such as schizophrenia, delusional disorders, affective disorders, autism or tic disorders, in particular chronic schizophrenic psychoses and schizoaffective psychoses, temporary acute psychotic disorders, depressive episodes, recurring depressive episodes, manic episodes and bipolar affective disorders.

Moreover, the invention is concerned with the use of a COX-2 inhibitor in combination with a neuroleptic drug or an antidepressant for the treatment of psychiatric disorders such as schizophrenia, delusional disorders, affective disorders, autism or tic disorders.

### BACKGROUND OF THE INVENTION

A relation between immunological dysfunctions and psychotic diseases, such as schizophrenia or affective disorders, has been discussed controversially over the last century.

In the case of schizophrenia for instance the pathogenesis is still unknown, but many findings indicate that schizophrenia is a syndrome based on different pathogenetic processes.

An inflammatory/immunological pathogenesis has been discussed for a subgroup of schizophrenic patients (Yolken R H, Torrey E F: Viruses, schizophrenia, and bipolar disorder. Clin Microbiol Rev 1995; 8: 131-145; Körschenhausen D, Hampel H, Ackenheil M, Penning R, Müller N: Fibrin degradation products in post mortem brain tissue of schizophrenics: a possible marker for underlying inflammatory processes, Schizophr Res 1996; 19: 103-109; Müller N, Ackenheil M: Psychoneuroimmunology and the cytokine-network in the CNS: implications for psychiatric disorders. Prog Neuropsychopharmacol & Biol Psychiat 1998; 22: 1-33). Studies showed that activating cytokines like interleukin-1 (IL-1) and IL-2 are increased in the cerebrospinal fluid of schizophrenic patients compared to controls (Sirota P, Schild K, Elizur A, Djaldetti M, Fishman P: Increased Interleukin-1 and Interleukin-3 like activity in schizophrenic patients. Prog Neuropsychopharmacol & Biol Psychiatry 1995; 19: 85-83; Licinio J, Seibyl, J P, Altemus M, Charney D S, Krystal J H: Elevated levels of Interleukin-2 in neuroleptic-free schizophrenics. Am J Psychiatry 1993; 150: 1408-1410), and that high levels of IL-2 in the cerebrospinal fluid are a predictor for the increased probability of a schizophrenic relapse (McAllister C G, van Kamen D P, Rehn T J, Miller A L, Gurklis J, Kelley M E, Yao J, Peters J L: Increases in CSF levels of Interleukin-2 in schizophrenia: effects of recurrence of psychosis and medication status. Am J Psychiatry 1995; 152: 1291-1297).

On the other hand, in a subgroup of schizophrenic patients a decreased immune response compared to controls has been observed, possibly due to a disturbance of antigen-presentation or antigen-recognition (Schwarz M J, Riedel M, Ackenheil M, Müller N: Decreased levels of soluble intercellular adhesion molecule-1 (sICAM-1) in unmedicated and medicated schizophrenic patients. Biol Psychiatry 2000; 47: 29-33), e.g. the increased immune reaction in the central nervous system may not be adequately regulated by an immune reaction in the peripheral immune system. This was observed mostly in acute schizophrenic patients presenting a recent onset of the disorder.

Another group of schizophrenic patients, however, seems to present an over-activation of the peripheral immune system in the sense of autoimmune processes (Radaport M H, Müller N: Immunological states associated with schizophrenia. In: Ader R, Felten D L, Cohen N (eds) Psychoneuroimmunology, Third Edition. Vol. 2, San Diego, Academic Press, 2001; pp 373-382; Radaport M H, McAllister C G, Kim Y S, Han J H, Pickar D, Nelson D M, Kirch D G, Paul S M: Increased soluble Interleukin-2 receptors in Caucasian and korean schizophrenic patients. Biol Psychiatry 1994; 35: 767-771). In several studies, increased titers of antibodies against the heat-shock-protein 60 were observed (Kilidireas K, Latov N, Strauss D H, Aviva D G, Hashim G A, Gorman J M, Sadiq S A: Antibodies to human 60 KD hear-shock protein in patients with schizophrenia. Lancet 1992; 340: 569-572), the increase being accompanied by increased soluble IL-2 receptors in the serum and increased titers of the soluble adhesion molecule sICAM-1 (Radaport M H, Müller N: Immunological states associated with schizophrenia. In: Ader R, Felten D L, Cohen N (eds) Psychoneuroimmunology, Third Edition. Vol. 2, San Diego, Academic Press, 2001; pp 373-382; Schwarz M J, Riedel M, Gruber R, Ackenheil M, Müller N: Antibodies to heat-shock proteins in schizophrenic patients-Implications for disease mechanism. Am J Psychiatry 1999; 156, 1103, 1104). The close relationship between high sVCAM-1 titers and more pronounced schizophrenic negative symptoms (Schwarz M J, Riedel M, Gruber R, Ackenheil M, Müller N: Levels of soluble adhesion molecules in schizophrenia: Relation to psychopathology. In: N. Müller (Hrg) Psychiatry, Psychoneuroimmunology, and Viruses. Springer Verlag Wien, 1999; NY, pp. 121-130) as well as between high IgG levels in the cerebrospinal fluid and more pronounced negative symptoms further support this observation (Müller N, Ackenheil M: Immunoglobulin and albumin contents of cerebrospinal fluid in schizophrenic patients: The relationship to negative sympomatology. Schizophrenia Res 1995; 14: 223-228).

Affective diseases, in particular depressive diseases, may also have an inflammatory genesis. This is manifested in the fact that general inflammatory diseases are accompanied by depressive syndromes to an increased extent as well as in the fact that in depressive diseases, signs of inflammation occur more frequently in comparison to psychologically healthy persons. Scientifically, this was expressed in the monocyte/macrophage hypothesis of depression.

The occurrence of tics as well as of autism has also been discussed in many cases as a consequence of inflammatory processes.

### DETAILED DESCRIPTION OF THE INVENTION

The invention is based on the idea that substances with immunomodulatory properties could be used for the treatment of psychiatric disorders such as schizophrenia, delusional disorders, affective disorders, autism or tic disorders, which are at least partially based on immunological pathogenetic processes.

For example, in the treatment of schizophrenia, a number of neuroleptic drugs (so-called classical and atypical neuroleptics) have become available, among which the more recent atypical neuroleptics excel by comparatively good effectiveness with a more favorable side effect profile. Unlike the classical neuroleptics, which are mainly effective for treating the positive symptoms of schizophrenia, the atypical neuroleptics improve both positive symptoms (hallucinations, delusions, and conceptual disorganization) and negative symptoms (apathy, social withdrawal, affective flattening, and poverty of speech) of schizophrenia. Plus, presumably due to their altered receptor binding profile, the atypical cause minimal extrapyramidal symptoms and rarely cause tardive dyskinesias.

Anyhow, neuroleptics in general act as syndrome oriented therapy and less as a causal therapy.

Therefore, a need exists for further medicaments for the treatment of psychiatric disorders such as schizophrenia, delusional disorders, affective disorders, autism or tic disorders.

The present invention is directed to the use of COX-2 inhibitors for the manufacture of a medicament for the treatment of psychiatric disorders such as schizophrenia, delusional disorders, affective disorders, autism or tic disorders, in particular chronic schizophrenic psychosis and schizoaffective psychosis, temporary acute psychotic disorders, depressive episodes, recurring depressive episodes, manic episodes and bipolar affective disorders.

In the context of the present invention a treatment of a disease or disorder is meant to cover the actual therapy as well as maintenance therapy and prophylaxis against recurrence.

Furthermore, the invention concerns the use of COX-2 inhibitors in combination with neuroleptics or antidepressants for the treatment of psychiatric disorders such as schizophrenia, delusional disorders, affective disorders, autism or tic disorders, in particular chronic schizophrenic psychosis and schizoaffective psychosis, temporary acute psychotic disorders, depressive episodes, recurring depressive episodes, manic episodes and bipolar affective disorders.

The invention is also directed to a novel kit-of-parts that is suitable for use in the treatment of psychiatric disorders such as schizophrenia, delusional disorders, affective disorders, autism or tic disorders, the kit comprising a first dosage form comprising a neuroleptic or an antidepressant and a second dosage form comprising a COX-2 inhibitor, for simultaneous, separate or sequential administration.

The COX-2 inhibitors of the present invention belong to the class of nonsteroidal anti-inflammatory drugs (NSAIDs). It has been known for some time that many of the common NSAIDs modulate prostaglandin synthesis by inhibition of cyclooxygenases that catalyze the transformation of arachidonic acid—the first step in the prostaglandin synthesis pathway. However, the use of high doses of many common NSAIDs can produce severe side effects that limit their therapeutic potential. In an effort to reduce the unwanted side effects of common NSAIDS, it was discovered that two cyclooxygenases are involved in the transformation of arachidonic acid as the first step in the prostaglandin synthesis pathway. These enzymes have been termed cyclooxygenase-1 (COX-1) and cyclooxygenase-2 (COX-2) (Needleman, P. et al., J. Rheumatol., 24, Suppl. 49: 6-8 (1997); Fu, J. Y., et al., J. Biol. Chem., 265(28): 16737-40 (1990)). COX-1 has been shown to be a constitutively produced enzyme that is involved in many of the non-inflammatory regulatory functions associated with prostaglandins. COX-2, on the other hand, is an inducible enzyme having significant involvement in the inflammatory process. Inflammation causes the induction of COX-2, leading to the release of prostanoids, which sensitize peripheral nociceptor terminals and produce localized pain hypersensitivity (Samad, T. A. et al., Nature, 410(6827): 471-5 (2001)). Many of the common NSAIDs are now known to be inhibitors of both COX-1 and COX-2. Accordingly, when administered in sufficiently high levels, these NSAIDs affect not only the inflammatory consequences of COX-2 activity, but also the beneficial activities of COX-1. Recently, compounds that selectively inhibit COX-2 to a greater extent than the activity of COX-1 have been discovered. These new COX-2 inhibitors are believed to offer advantages that include the capacity to prevent or reduce inflammation while avoiding harmful side effects associated with the inhibition of COX-1, such as gastrointestinal and renal side effects, as well as inhibition of thrombocyte aggregation.

The use of COX-2 inhibitors in the therapy of arthritis and related indications is known. U.S. Pat. No. 5,760,068 describes the use of COX-2 inhibitors for the treatment of rheumatoid arthritis and osteoarthritis. WO 00/32189 discloses the preparation of pharmaceutical compositions containing the COX-2 inhibitor celecoxib and the use of celecoxib for the treatment of rheumatoid arthritis or as a painkiller.

The term COX-2 inhibitor embraces compounds which selectively inhibit cyclooxygenase-2 over cyclooxygenase-1, and also includes pharmaceutically acceptable salts thereof. Also included within the scope of the present invention are compounds that act as prodrugs of cyclooxygenase-2-selective inhibitors. As used herein in reference to COX-2 inhibitors, the term “prodrug” refers to a chemical compound that can be converted into an active COX-2 inhibitor by metabolic or simple chemical processes within the body of the subject.

The COX-2 inhibitor of the present invention can be, for example, the COX-2 inhibitor meloxicam, Formula B-1 (CAS registry number 71125-38-7), or a pharmaceutical acceptable salt or prodrug thereof.

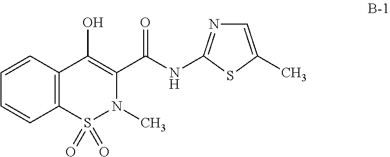

In another embodiment of the invention the COX-2 inhibitor can be the COX-2 inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

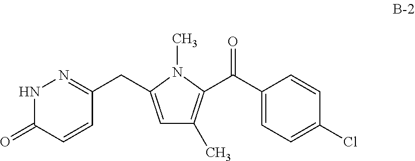

In a preferred embodiment of the invention the COX-2 inhibitor is a chromene derivative, that is a substituted benzopyran or a substituted benzopyran analog, and even more preferably selected from the group consisting of substituted benzothiopyrans, dihydroquinolines, or dihydronaphthalenes having the structure of any one of the compounds having a structure shown by general Formulas I, II, or III, shown below, and possessing, by way of example and not limitation, the structures disclosed in Table 1, including the diastereomers, enantiomers, racemates, tautomers, salts, esters, amides and prodrugs thereof.

Benzopyran COX-2 inhibitors useful in the practice of the present invention are described in U.S. Pat. Nos. 6,034,256 and 6,077,850.
Formula I is:

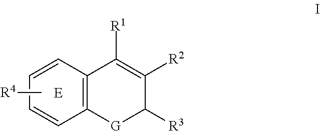

wherein G is selected from the group consisting of O or S or NR^{a};
wherein R^{a} is alkyl;
wherein R¹ is selected from the group consisting of H and aryl;
wherein R² is selected from the group consisting of carboxyl, aminocarbonyl, alkylsulfonylaminocarbonyl and alkoxycarbonyl;
wherein R³ is selected from the group consisting of haloalkyl, alkyl, aralkyl, cycloalkyl and aryl optionally substituted with one or more radicals selected from alkylthio, nitro and alkylsulfonyl; and
wherein R⁴ is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;
or wherein R⁴ together with ring E forms a naphthyl radical; or an isomer or pharmaceutically acceptable salt thereof; and
   including the diastereomers, enantiomers, racemates, tautomers, salts, esters, amides and prodrugs thereof.

Formula II is:

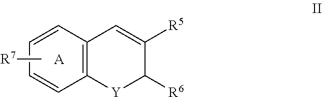

wherein:
Y is selected from the group consisting of O or S or NR^{b};
R^{b} is alkyl;
R⁵ is selected from the group consisting of carboxyl, aminocarbonyl, alkylsulfonylaminocarbonyl and alkoxycarbonyl;
R⁶ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
R⁷ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁷ together with ring A forms a naphthyl radical;
   or an isomer or pharmaceutical acceptable salt thereof.

The COX-2 inhibitor may also be a compound of Formula II, wherein:
Y is selected from the group consisting of oxygen and sulfur;
R⁵ is selected from the group consisting of carboxyl, lower alkyl, lower aralkyl and lower alkoxycarbonyl;
R⁶ is selected from the group consisting of lower haloalkyl, lower cycloalkyl and phenyl; and
R⁷ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or
wherein R⁷ together with ring A forms a naphthyl radical; or an isomer or pharmaceutical acceptable salt thereof.

The COX-2 inhibitor may also be a compound of Formula II, wherein:
R⁵ is carboxyl;
R⁶ is lower haloalkyl; and
R⁷ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R⁷ together with ring A forms a naphthyl radical;
or an isomer or pharmaceutical acceptable salt thereof.

The COX-2 inhibitor may also be a compound of Formula II, wherein:
R⁶ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and
R⁷ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or wherein R² together with ring A forms a naphthyl radical;
or an isomer or pharmaceutically acceptable salt thereof.

The COX-2 inhibitor may also be a compound of Formula II, wherein:
R⁶ is selected from the group consisting trifluoromethyl and pentafluoroethyl; and
R⁷ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R² together with ring A forms a naphthyl radical;
   or an isomer or prodrug thereof.

The COX-2 inhibitor of the present invention can also be a compound having the structure of Formula III:

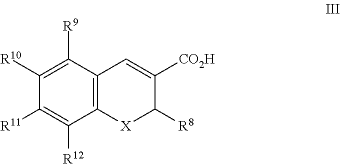

wherein:
X is selected from the group consisting of O and S;
R⁸ is lower haloalkyl;
R⁹ is selected from the group consisting of hydrido, and halo;
R¹⁰ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;
R¹¹ is selected from the group consisting of hydrido, lower alkyl, halo, lower alkoxy, and aryl; and
R¹² is selected from the group consisting of the group consisting of hydrido, halo, lower alkyl, lower alkoxy, and aryl; or an isomer or prodrug thereof.

The COX-2 inhibitor can also be a compound of having the structure of Formula III, wherein
R⁸ is selected from the group consisting of trifluoromethyl and pentafluoroethyl;
R⁹ is selected from the group consisting of hydrido, chloro, and fluoro;
R¹⁰ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;
R¹¹ is selected from the group consisting of hydrido, methyl, ethyl, isopropyl, tert-butyl, chloro, methoxy, diethylamino, and phenyl; and
R¹² is selected from the group consisting of hydrido, chloro, bromo, fluoro, methyl, ethyl, tert-butyl, methoxy, and phenyl; or an isomer or prodrug thereof.

TABLE 1
Examples of Chromene COX-2 Inhibitors as Embodiments

| Compound | |
|---|---|
| Number | Structural Formula |
| B-3 | 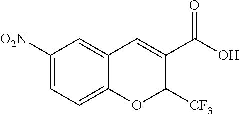 |
| | 6-Nitro-2-trifluoromethyl-2H-1- |
| | benzopyran-3-carboxylic acid |
| B-4 | 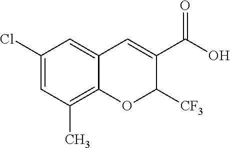 |
| | 6-Chloro-8-methyl-2-trifluoromethyl-2H- |
| | 1-benzopyran-3-carboxylic acid |
| B-5 | 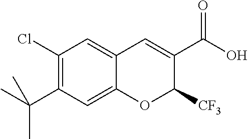 |
| | ((S)-6-Chloro-7-(1,1-dimethylethyl)-2- |
| | (trifluo romethyl-2H-1-benzopyran-3- |
| | carboxylic acid |
| B-6 | 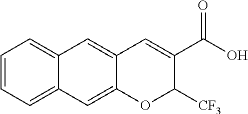 |
| | 2-Trifluoromethyl-2H-naphtho [2,3-b] |
| | pyran-3-carboxylic acid |
| B-7 | 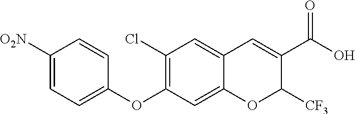 |
| | 6-Chloro-7-(4-nitrophenoxy)-2- |
| | (trifluoromethyl)-2H-1-benzopyran-3- |
| | carboxylic acid |
| B-8 | 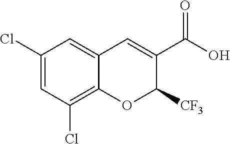 |
| | ((S)-6,8-Dichloro-2-(trifluoromethyl)-2H- |
| | 1-benzopyran-3-carboxylic acid |
| B-9 | 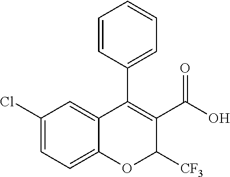 |
| | 6-Chloro-2-(trifluoromethyl)-4-phenyl- |
| | 2H-1-benzopyran-3-carboxylic acid |
| B-10 | 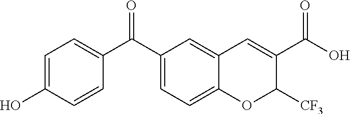 |
| | 6-(4-Hydroxybenzoyl)-2- |
| | (trifluoromethyl)-2H-1-benzopyran-3- |
| | carboxylic acid |
| B-11 | 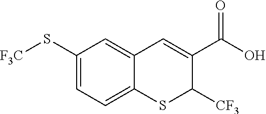 |
| | 2-(Trifluoromethyl)-6-[(trifluoromethyl) |
| | thio]-2H-1-benzothiopyran-3-carboxylic |
| | acid |
| B-12 | 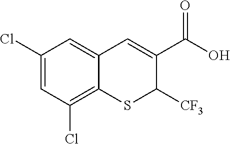 |
| | 6,8-Dichloro-2-trifluoromethyl-2H-1- |
| | benzothiopyran-3-carboxylic acid |
| B-13 | 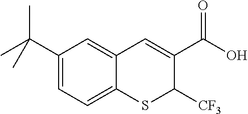 |
| | 6-(1,1-Dimethylethyl)-2- |
| | (trifluoromethyl)-2H-1-benzothiopyran- |
| | 3-carboxylic acid |
| B-14 | 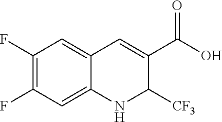 |
| | 6,7-Difluoro-1,2-dihydro-2-(trifluoro |
| | methyl)-3-quinolinecarboxylic acid |
| B-15 | 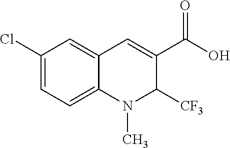 |
| | 6-Chloro-1,2-dihydro-1-methyl-2- |
| | (trifluoro methyl)-3-quinolinecarboxylic |
| | acid |
| B-16 | 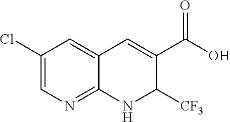 |
| | 6-Chloro-2-(trifluoromethyl)-1,2-dihydro |
| | [1,8] naphthyridine-3-carboxylic acid |
| B-17 | 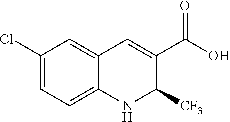 |
| | ((S)-6-Chloro-1,2-dihydro-2-(trifluoro |
| | methyl)-3-quinolinecarboxylic acid |

Specific compounds that are useful for the COX-2 inhibitor include:

a1) 8-acetyl-3-(4-fluorophenyl)-2-(4-methylsulfonyl)phenyl-imidazo (1,2-a) pyridine;
a2) 5,5-dimethyl-4-(4-methylsulfonyl)phenyl-3-phenyl-2-(5H)-furanone;
a3) 5-(4-fluorophenyl)-1-[4-(methylsulfonyl)phenyl]-3-(trifluoromethyl)pyrazole;
a4) 4-(4-fluorophenyl)-5-[4-(methylsulfonyl)phenyl]-1-phenyl-3-(trifluoromethyl)pyrazole;
a5) 4-(5-(4-chlorophenyl)-3-(4-methoxyphenyl)-1H-pyrazol-1-yl)benzenesulfonamide;
a6) 4-(3,5-bis(4-methylphenyl)-1H-pyrazol-1-yl)benzenesulfonamide;
a7) 4-(5-(4-chlorophenyl)-3-phenyl-1H-pyrazol-1-yl)benzenesulfonamide;
a8) 4-(3,5-bis(4-methoxyphenyl)-1H-pyrazol-1-yl)benzenesulfonamide;
a9) 4-(5-(4-chlorophenyl)-3-(4-methylphenyl)-1H-pyrazol-1-yl)benzenesulfonamide;
a10) 4-(5-(4-chlorophenyl)-3-(4-nitrophenyl)-1H-pyrazol-1-yl)benzenesulfonamide;
b1) 4-(5-(4-chlorophenyl)-3-(5-chloro-2-thienyl)-1H-pyrazol-1-yl)benzenesulfonamide;
b2) 4-(4-chloro-3,5-diphenyl-1H-pyrazol-1-yl)benzenesulfonamide
b3) 4-[5-(4-chlorophenyl)-3-(trifluoromethyl)-1H-pyrazol-1-yl]benzenesulfonamide;
b4) 4-[5-phenyl-3-(trifluoromethyl)-1H-pyrazol-1-yl]benzenesulfonamide;
b5) 4-[5-(4-fluorophenyl)-3-(trifluoromethyl)-1H-pyrazol-1-yl]benzenesulfonamide;
b6) 4-[5-(4-methoxyphenyl)-3-(trifluoromethyl)-1H-pyrazol-1-yl]benzenesulfonamide;
b7) 4-[(5-(4-chlorophenyl)-3-(difluoromethyl)-1H-pyrazol-1-yl]benzenesulfonamide;
b8) 4-[5-(4-methylphenyl)-3-(trifluoromethyl)-1H-pyrazol-1-yl]benzenesulfonamide;
b9) 4-[4-chloro-5-(4-chlorophenyl)-3-(trifluoromethyl)-1H-pyrazol-1-yl]benzenesulfonamide;
b10) 4-[3-(difluoromethyl)-5-(4-methylphenyl)-1H-pyrazol-1-yl]benzenesulfonamide;
c1) 4-[3-(difluoromethyl)-5-phenyl-1H-pyrazol-1-yl]benzenesulfonamide;
c2) 4-[3-(difluoromethyl)-5-(4-methoxyphenyl)-1H-pyrazol-1-yl]benzenesulfonamide;
c3) 4-[3-cyano-5-(4-fluorophenyl)-1H-pyrazol-1-yl]benzenesulfonamide,
c4) 4-[3-(difluoromethyl)-5-(3-fluoro-4-methoxyphenyl)-1H-pyrazol-1-yl]benzenesulfonamide;
c5) 4-[5-(3-fluoro-4-methoxyphenyl)-3-(trifluoromethyl)-1H-pyrazol-1-yl]benzenesulfonamide;
c6) 4-[4-chloro-5-phenyl-1H-pyrazol-1-yl]benzenesulfonamide;
c7) 4-[5-(4-chlorophenyl)-3-(hydroxymethyl)-1H-pyrazol-1-yl]benzenesulfonamide;
c8) 4-[5-(4-(N,N-dimethylamino)phenyl)-3-(trifluoromethyl)-1H-pyrazol-1-yl]benzenesulfonamide;
c9) 5-(4-fluorophenyl)-6-[4-(methylsulfonyl)phenyl]spiro[2.4]hept-5-ene;
c10) 4-[6-(4-fluorophenyl)spiro[2.4]hept-5-en-5-yl]benzenesulfonamide;
d1) 6-(4-fluorophenyl)-7-[4-(methylsulfonyl)phenyl]spiro[3.4]oct-6-ene;
d2) 5-(3-chloro-4-methoxyphenyl)-6-[4-(methylsulfonyl)phenyl]spiro[2.4]hept-5-ene;
d3) 4-[6-(3-chloro-4-methoxyphenyl)spiro[2.4]hept-5-en-5-yl]benzenesulfonamide;
d4) 5-(3,5-dichloro-4-methoxyphenyl)-6-[4-(methylsulfonyl)phenyl]spiro[2.4]hept-5-ene;
d5) 5-(3-chloro-4-fluorophenyl)-6-[4-(methylsulfonyl)phenyl]spiro[2.4]hept-5-ene;
d6) 4-[6-(3,4-dichlorophenyl)spiro[2.4]hept-5-en-5-yl]benzenesulfonamide;
d7) 2-(3-chloro-4-fluorophenyl)-4-(4-fluorophenyl)-5-(4-methylsulfonylphenyl)thiazole;
d8) 2-(2-chlorophenyl)-4-(4-fluorophenyl)-5-(4-methylsulfonylphenyl)thiazole;
d9) 5-(4-fluorophenyl)-4-(4-methylsulfonylphenyl)-2-methylthiazole;
d10) 4-(4-fluorophenyl)-5-(4-methylsulfonylphenyl)-2-trifluoromethylthiazole;
e1) 4-(4-fluorophenyl)-5-(4-methylsulfonylphenyl)-2-(2-thienyl)thiazole;
e2) 4-(4-fluorophenyl)-5-(4-methylsulfonylphenyl)-2-benzylaminothiazole;
e3) 4-(4-fluorophenyl)-5-(4-methylsulfonylphenyl)-2-(1-propylamino)thiazole;
e4) 2-[(3,5-dichlorophenoxy)methyl)-4-(4-fluorophenyl)-5-[4-(methylsulfonyl)phenyl]thiazole;
e5) 5-(4-fluorophenyl)-4-(4-methylsulfonylphenyl)-2-trifluoromethylthiazole;
e6) 1-methylsulfonyl-4-[1,1-dimethyl-4-(4-fluorophenyl)cyclopenta-2,4-dien-3-yl]benzene;
e7) 4-[4-(4-fluorophenyl)-1,1-dimethylcyclopenta-2,4-dien-3-yl]benzenesulfonamide;
e8) 5-(4-fluorophenyl)-6-[4-(methylsulfonyl)phenyl]spiro[2.4]hepta-4,6-diene;
e9) 4-[6-(4-fluorophenyl)spiro[2.4]hepta-4,6-dien-5-yl]benzenesulfonamide;
e10) 6-(4-fluorophenyl)-2-methoxy-5-[4-(methylsulfonyl)phenyl]-pyridine-3-carbonitrile;
f1) 2-bromo-6-(4-fluorophenyl)-5-[4-(methylsulfonyl)phenyl]-pyridine-3-carbonitrile;
f2) 6-(4-fluorophenyl)-5-[4-(methylsulfonyl)phenyl]-2-phenyl-pyridine-3-carbonitrile;
f3) 4-[2-(4-methylpyridin-2-yl)-4-(trifluoromethyl)-1H-imidazol-1-yl]benzenesulfonamide;
f4) 4-[2-(5-methylpyridin-3-yl)-4-(trifluoromethyl)-1H-imidazol-1-yl]benzenesulfonamide;
f5) 4-[2-(2-methylpyridin-3-yl)-4-(trifluoromethyl)-1H-imidazol-1-yl]benzenesulfonamide;
f6) 3-[1-[4-(methylsulfonyl)phenyl]-4-(trifluoromethyl)-1H-imidazol-2-yl]pyridine;
f7) 2-[1-[4-(methylsulfonyl)phenyl-4-(trifluoromethyl)-1H-imidazol-2-yl]pyridine;
f8) 2-methyl-4-[1-[4-(methylsulfonyl)phenyl-4-(trifluoromethyl)-1H-imidazol-2-yl]pyridine;
f9) 2-methyl-6-[1-[4-(methylsulfonyl)phenyl-4-(trifluoromethyl)-1H-imidazol-2-yl]pyridine;
f10) 4-[2-(6-methylpyridin-3-yl)-4-(trifluoromethyl)-1H-imidazol-1-yl]benzenesulfonamide;
g1) 2-(3,4-difluorophenyl)-1-[4-(methylsulfonyl)phenyl]-4-(trifluoromethyl)-1H-imidazole;
g2) 4-[2-(4-methylphenyl)-4-(trifluoromethyl)-1H-imidazol-1-yl]benzenesulfonamide;
g3) 2-(4-chlorophenyl)-1-[4-(methylsulfonyl)phenyl]-4-methyl-1H-imidazole;
g4) 2-(4-chlorophenyl)-1-[4-(methylsulfonyl)phenyl]-4-phenyl-1H-imidazole;
g5) 2-(4-chlorophenyl)-4-(4-fluorophenyl)-1-[4-(methylsulfonyl)phenyl]-1H-imidazole;
g6) 2-(3-fluoro-4-methoxyphenyl)-1-[4-(methylsulfonyl)phenyl-4-(trifluoromethyl)-1H-imidazole;
g7) 1-[4-(methylsulfonyl)phenyl]-2-phenyl-4-trifluoromethyl-1H-imidazole;
g8) 2-(4-methylphenyl)-1-[4-(methylsulfonyl)phenyl]-4-trifluoromethyl-1H-imidazole;
g9) 4-[2-(3-chloro-4-methylphenyl)-4-(trifluoromethyl)-1H-imidazol-1-yl]benzenesulfonamide;
g10) 2-(3-fluoro-5-methylphenyl)-1-[4-(methylsulfonyl)phenyl]-4-(trifluoromethyl)-1H-imidazole;
h1) 4-[2-(3-fluoro-5-methylphenyl)-4-(trifluoromethyl)-1H-imidazol-1-yl]benzenesulfonamide;
h2) 2-(3-methylphenyl)-1-[4-(methylsulfonyl)phenyl]-4-trifluoromethyl-1H-imidazole;
h3) 4-[2-(3-methylphenyl)-4-trifluoromethyl-1H-imidazol-1-yl]benzenesulfonamide;
h4) 1-[4-(methylsulfonyl)phenyl]-2-(3-chlorophenyl)-4-trifluoromethyl-1H-imidazole;
h5) 4-[2-(3-chlorophenyl)-4-trifluoromethyl-1H-imidazol-1-yl]benzenesulfonamide;
h6) 4-[2-phenyl-4-trifluoromethyl-1H-imidazol-1-yl]benzenesulfonamide;
h7) 4-[2-(4-methoxy-3-chlorophenyl)-4-trifluoromethyl-1H-imidazol-1-yl]benzenesulfonamide;
h8) 1-allyl-4-(4-fluorophenyl)-3-[4-(methylsulfonyl)phenyl]-5-(trifluoromethyl)-1H-pyrazole;
h10) 4-[1-ethyl-4-(4-fluorophenyl)-5-(trifluoromethyl)-1H-pyrazol-3-yl]benzenesulfonamide;
i1) N-phenyl-[4-(4-luorophenyl)-3-[4-(methylsulfonyl)phenyl]-5-(trifluoromethyl)-1H-pyrazol-1-yl]acetamide;
i2) ethyl [4-(4-fluorophenyl)-3-[4-(methylsulfonyl)phenyl]-5-(trifluoromethyl)-1H-pyrazol-1-yl]acetate;
i3) 4-(4-fluorophenyl)-3-[4-(methylsulfonyl)phenyl]-1-(2-phenylethyl)-1H-pyrazole;
i4) 4-(4-fluorophenyl)-3-[4-(methylsulfonyl)phenyl]-1-(2-phenylethyl)-5-(trifluoromethyl)pyrazole;
i5) 1-ethyl-4-(4-fluorophenyl)-3-[4-(methylsulfonyl)phenyl]-5-(trifluoromethyl)-1H-pyrazole;
i6) 5-(4-fluorophenyl)-4-(4-methylsulfonylphenyl)-2-trifluoromethyl-1H-imidazole;
i7) 4-[4-(methylsulfonyl)phenyl]-5-(2-thiophenyl)-2-(trifluoromethyl)-1H-imidazole;
i8) 5-(4-fluorophenyl)-2-methoxy-4-[4-(methylsulfonyl)phenyl]-6-(trifluoromethyl)pyridine;
i9) 2-ethoxy-5-(4-fluorophenyl)-4-[4-(methylsulfonyl)phenyl]-6-(trifluoromethyl)pyridine;
i10) 5-(4-fluorophenyl)-4-[4-(methylsulfonyl)phenyl]-2-(2-propynyloxy)-6-(trifluoromethyl)pyridine;
j1) 2-bromo-5-(4-fluorophenyl)-4-[4-(methylsulfonyl)phenyl]-6-(trifluoromethyl)pyridine;
j2) 4-[2-(3-chloro-4-methoxyphenyl)-4,5-difluorophenyl]benzenesulfonamide;
j3) 1-(4-fluorophenyl)-2-[4-(methylsulfonyl)phenyl]benzene;
j4) 5-difluoromethyl-4-(4-methylsulfonylphenyl)-3-phenylisoxazole;
j5) 4-[3-ethyl-5-phenylisoxazole-4-yl]benzenesulfonamide;
j6) 4-[5-difluoromethyl-3-phenylisoxazole-4-yl]benzenesulfonamide;
j7) 4-[5-hydroxymethyl-3-phenylisoxazole-4-yl]benzenesulfonamide;
j8) 4-[5-methyl-3-phenyl-isoxazol-4-yl]benzenesulfonamide;
j9) 1-[2-(4-fluorophenyl)cyclopenten-1-yl]-4-(methylsulfonyl)benzene;
j10) 1-[2-(4-fluoro-2-methylphenyl)cyclopenten-1-yl]-4-(methylsulfonyl)benzene;
k1) 1-[2-(4-chlorophenyl)cyclopenten-1-yl]-4-(methylsulfonyl)benzene;
k2) 1-[2-(2,4-dichlorophenyl)cyclopenten-1-yl]-4-(methylsulfonyl)benzene;
k3) 1-[2-(4-trifluoromethylphenyl)cyclopenten-1-yl]-4-(methylsulfonyl)benzene;
k4) 1-[2-(4-methylthiophenyl)cyclopenten-1-yl]-4-(methylsulfonyl)benzene;
k5) 1-[2-(4-fluorophenyl)-4,4-dimethylcyclopenten-1-yl]-4-(methylsulfonyl)benzene;
k6) 4-[2-(4-fluorophenyl)-4,4-dimethylcyclopenten-1-yl]benzenesulfonamide;
k7) 1-[2-(4-chlorophenyl)-4,4-dimethylcyclopenten-1-yl]-4-(methylsulfonyl)benzene;
k8) 4-[2-(4-chlorophenyl)-4,4-dimethylcyclopenten-1-yl]benzenesulfonamide;
k9) 4-[2-(4-fluorophenyl)cyclopenten-1-yl]benzenesulfonamide;
k10) 4-[2-(4-chlorophenyl)cyclopenten-1-yl]benzenesulfonamide;
l1) 1-[2-(4-methoxyphenyl)cyclopenten-1-yl]-4-(methylsulfonyl)benzene;
l2) 1-[2-(2,3-difluorophenyl)cyclopenten-1-yl]-4-(methylsulfonyl)benzene;
l3) 4-[2-(3-fluoro-4-methoxyphenyl)cyclopenten-1-yl]benzenesulfonamide;
l4) 1-[2-(3-chloro-4-methoxyphenyl)cyclopenten-1-yl]-4-(methylsulfonyl)benzene;
l5) 4-[2-(3-chloro-4-fluorophenyl)cyclopenten-1-yl]benzenesulfonamide;
l6) 4-[2-(2-methylpyridin-5-yl)cyclopenten-1-yl]benzenesulfonamide;
l7) ethyl 2-[4-(4-fluorophenyl)-5-[4-(methylsulfonyl)phenyl]oxazole-2-yl]-2-benzyl-acetate;
l8) 2-[4-(4-fluorophenyl)-5-[4-(methylsulfonyl)phenyl]oxazol-2-yl]acetic acid;
l9) 2-(tert-butyl)-4-(4-fluorophenyl)-5-[4-(methylsulfonyl)phenyl]oxazole;
l10) 4-(4-fluorophenyl)-5-[4-(methylsulfonyl)phenyl]-2-phenyloxazole;
m1) 4-(4-fluorophenyl)-2-methyl-5-[4-(methylsulfonyl)phenyl]oxazole; and
m2) 4-[5-(3-fluoro-4-methoxyphenyl)-2-trifluoromethyl-4-oxazolyllbenzenesulfonamide.
m3) 6-chloro-2-trifluoromethyl-2H-1-benzopyran-3-carboxylic acid;
m4) 6-chloro-7-methyl-2-trifluoromethyl-2H-1-benzopyran-3-carboxylic acid;
m5) 8-(1-methylethyl)-2-trifluoromethyl-2H-1-benzopyran-3-carboxylic acid;
m6) 6-chloro-7-(1,1-dimethylethyl)-2-trifluoromethyl-2H-1-benzopyran-3-carboxylic acid;
m7) 6-chloro-8-(1-methylethyl)-2-trifluoromethyl-2H-1-benzopyran-3-carboxylic acid;
m8) 2-trifluoromethyl-3H-naphthopyran-3-carboxylic acid;
m9) 7-(1,1-dimethylethyl)-2-trifluoromethyl-2H-1-benzopyran-3-carboxylic acid;
m10) 6-bromo-2-trifluoromethyl-2H-1-benzopyran-3-carboxylic acid;
n1) 8-chloro-2-trifluoromethyl-2H-1-benzopyran-3-carboxylic acid;
n2) 6-trifluoromethoxy-2-trifluoromethyl-2H-1-benzopyran-3-carboxylic acid;
n3) 5,7-dichloro-2-trifluoromethyl-2H-1-benzopyran-3-carboxylic acid;
n4) 8-phenyl-2-trifluoromethyl-2H-1-benzopyran-3-carboxylic acid;
n5) 7,8-dimethyl-2-trifluoromethyl-2H-1-benzopyran-3-carboxylic acid;
n6) 6,8-bis(dimethylethyl)-2-trifluoromethyl-2H-1-benzopyran-3-carboxylic acid;
n7) 7-(1-methylethyl)-2-trifluoromethyl-2H-1-benzopyran-3-carboxylic acid;
n8) 7-phenyl-2-trifluoromethyl-2H-1-benzopyran-3-carboxylic acid;
n9) 6-chloro-7-ethyl-2-trifluoromethyl-2H-1-benzopyran-3-carboxylic acid;
n10) 6-chloro-8-ethyl-2-trifluoromethyl-2H-1-benzopyran-3-carboxylic acid;
o1) 6-chloro-7-phenyl-2-trifluoromethyl-2H-1-benzopyran-3-carboxylic acid;
o2) 6,7-dichloro-2-trifluoromethyl-2H-1-benzopyran-3-carboxylic acid;
o3) 6,8-dichloro-2-trifluoromethyl-2H-1-benzopyran-3-carboxylic acid;
o4) 2-trifluoromethyl-3H-naptho[2,1-b]pyran-3-carboxylic acid;
o5) 6-chloro-8-methyl-2-trifluoromethyl-2H-1-benzopyran-3-carboxylic acid;
o6) 8-chloro-6-methyl-2-trifluoromethyl-2H-1-benzopyran-3-carboxylic acid;
o7) 8-chloro-6-methoxy-2-trifluoromethyl-2H-1-benzopyran-3-carboxylic acid;
o8) 6-bromo-8-chloro-2-trifluoromethyl-2H-1-benzopyran-3-carboxylic acid;
o9) 8-bromo-6-fluoro-2-trifluoromethyl-2H-1-benzopyran-3-carboxylic acid;
o10) 8-bromo-6-methyl-2-trifluoromethyl-2H-1-benzopyran-3-carboxylic acid;
p1) 8-bromo-5-fluoro-2-trifluoromethyl-2H-1-benzopyran-3-carboxylic acid;
p2) 6-chloro-8-fluoro-2-trifluoromethyl-2H-1-benzopyran-3-carboxylic acid;
p3) 6-bromo-8-methoxy-2-trifluoromethyl-2H-1-benzopyran-3-carboxylic acid;
p4) 6-[[(phenylmethyl)amino]sulfonyl]-2-trifluoromethyl-2H-1-benzopyran-3-carboxylic acid;
p5) 6-[(dimethylamino)sulfonyl]-2-trifluoromethyl-2H-1-benzopyran-3-carboxylic acid;
p6) 6-[(methylamino)sulfonyl]-2-trifluoromethyl-2H-1-benzopyran-3-carboxylic acid;
p7) 6-[(4-morpholino)sulfonyl]-2-trifluoromethyl-2H-1-benzopyran-3-carboxylic acid;
p8) 6-[(1,1-dimethylethyl)aminosulfonyl]-2-trifluoromethyl-2H-1-benzopyran-3-carboxylic acid;
p9) 6-[(2-methylpropyl)aminosulfonyl]-2-trifluoromethyl-2H-1-benzopyran-3-carboxylic acid;
p10) 6-methylsulfonyl-2-trifluoromethyl-2H-1-benzopyran-3-carboxylic acid;
q1) 8-chloro-6-[[(phenylmethyl)amino]sulfonyl]-2-trifluoromethyl-2H-1-benzopyran-3-carboxylic acid;
q2) 6-phenylacetyl-2-trifluoromethyl-2H-1-benzopyran-3-carboxylic acid;
q3) 6,8-dibromo-2-trifluoromethyl-2H-1-benzopyran-3-carboxylic acid;
q4) 8-chloro-5,6-dimethyl-2-trifluoromethyl-2H-1-benzopyran-3-carboxylic acid;
q5) 6,8-dichloro-(S)-2-trifluoromethyl-2H-1-benzopyran-3-carboxylic acid;
q6) 6-benzylsulfonyl-2-trifluoromethyl-2H-1-benzopyran-3-carboxylic acid;
q7) 6-[[N-(2-furylmethyl)amino]sulfonyl]-2-trifluoromethyl-2H-1-benzopyran-3-carboxylic acid;

q8) 6-[[N-(2-phenylethyl)amino]sulfonyl]-2-trifluoromethyl-2H-1-benzopyran-3-carboxylic acid;

q9) 6-iodo-2-trifluoromethyl-2H-1-benzopyran-3-carboxylic acid;
q10) 7-(1,1-dimethylethyl)-2-pentafluoroethyl-2H-1-benzopyran-3-carboxylic acid;
r1) 5,5-dimethyl-3-(3-fluorophenyl)-4-(4-methyl-sulphonyl-2(5H)-fluranone;
r2) 6-chloro-2-trifluoromethyl-2H-1-benzothiopyran-3-carboxylic acid;
r3) 4-[5-(4-chlorophenyl)-3-(trifluoromethyl)-1H-pyrazol-1-yl]benzenesulfonamide;
r4) 4-[5-(4-methylphenyl)-3-(trifluoromethyl)-1H-pyrazol-1-yl]benzenesulfonamide;
r5) 4-[5-(3-fluoro-4-methoxyphenyl)-3-(difluoromethyl)-1H-pyrazol-1-yl]benzenesulfonamide;
r6) 3-[1-[4-(methylsulfonyl)phenyl]-4-trifluoromethyl-1H-imidazol-2-yl]pyridine;
r7) 2-methyl-5-[1-[4-(methylsulfonyl)phenyl]-4-trifluoromethyl-1H-imidazol-2-yl]pyridine,
r8) 4-[2-(5-methylpyridin-3-yl)-4-(trifluoromethyl)-1H-imidazol-1-yl]benzenesulfonamide;
r9) 4-[5-methyl-3-phenylisoxazol-4-yl]benzenesulfonamide;
r10) 4-[5-hydroxymethyl-3-phenylisoxazol-4-yl]benzenesulfonamide;
s1) [2-trifluoromethyl-5-(3,4-difluorophenyl)-4-oxazolyl]benzenesulfonamide;
s2) 4-[2-methyl-4-phenyl-5-oxazolyl]benzenesulfonamide; or
s3) 4-[5-(3-fluoro-4-methoxyphenyl-2-trifluoromethyl)-4-oxazolyl]benzenesulfonamide;
or a pharmaceutically acceptable salt or prodrug thereof.

In a further preferred embodiment of the invention the cyclooxygenase inhibitor can be selected from the class of tricyclic COX-2 inhibitors represented by the general structure of Formula IV:

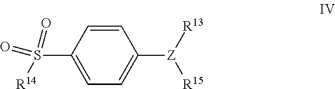

wherein:
Z is selected from the group consisting of partially unsaturated or unsaturated heterocyclyl and partially unsaturated or unsaturated carbocyclic rings;
R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R¹⁴ is selected from the group consisting of methyl or amino; and
R¹⁵ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;
or a prodrug thereof.

In a preferred embodiment of the invention the COX-2 inhibitor represented by the above Formula IV is selected from the group of compounds, illustrated in Table 2, which includes celecoxib (B-18), valdecoxib (B-19), deracoxib (B-20), rofecoxib (B-21), etoricoxib (MK-663; B-22), JTE-522 (B-23), or a prodrug thereof.

Additional information about selected examples of the COX-2 inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 2
Examples of Tricyclic COX-2 Inhibitors as Embodiments

| Compound | |
|---|---|
| Number | Structural Formula |
| B-18 | 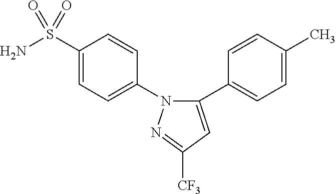 |
| B-19 | 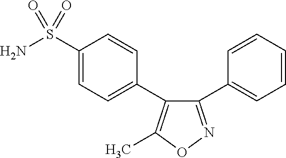 |
| B-20 | 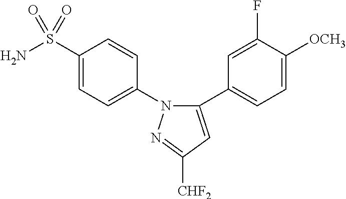 |
| B-21 | 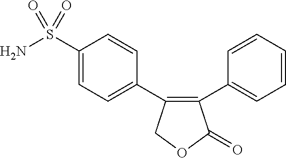 |
| B-22 | 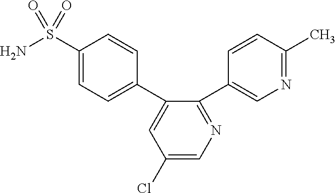 |
| B-23 | 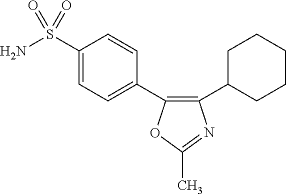 |

In a more preferred embodiment of the invention, the COX-2 inhibitor is selected from the group consisting of celecoxib, rofecoxib, etoricoxib and cimicoxib. In a preferred embodiment of the invention, parecoxib (U.S. Pat. No. 5,932,598), having the structure shown in B-24, which is a therapeutical effective prodrug of the tricyclic COX-2 inhibitor valdecoxib, B-19, (U.S. Pat. No. 5,633,272), may be advantageously employed as a source of a cyclooxygenase inhibitor. A preferred form of parecoxib is sodium parecoxib.

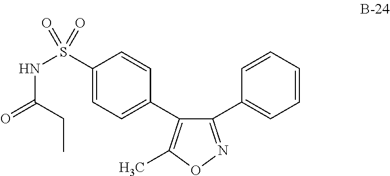

In another preferred embodiment of the invention, the compound ABT-963 having the formula B-25 that has been previously described in International Publication number WO 00/24719, is another tricyclic COX-2 inhibitor which may be advantageously employed.

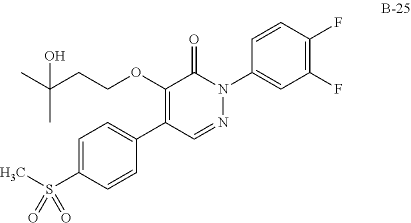

In a further preferred embodiment of the invention the cyclooxygenase inhibitor can be selected from the class of phenylacetic acid derivative COX-2 inhibitors represented by the general structure of Formula V:

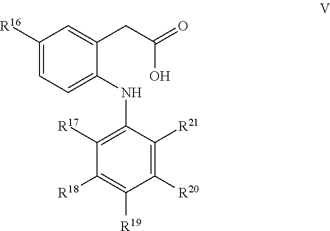

wherein R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R¹⁹ and R²⁰ are not all fluoro when R¹⁶ is ethyl and R¹⁹ is H.

A particularly preferred phenylacetic acid derivative COX-2 inhibitor that is described in WO 99/11605 is a compound that has the designation of COX189 (CAS RN 346670-74-4), and that has the structure shown in Formula V,
wherein R¹⁶ is ethyl;
R¹⁷ and R¹⁹ are chloro;
R¹⁸ and R²⁰ are hydrogen;
and R²¹ is methyl.

Compounds that have a structure similar to that shown in Formula V, which can serve as the COX-2 inhibitor of the present invention, are described in U.S. Pat. Nos. 6,310,099 and 6,291,523.

Other preferred COX-2 inhibitors that can be used in the present invention have the general structure shown in formula VI, where the J group is a carbocycle or a heterocycle. Particularly preferred embodiments have the structure:

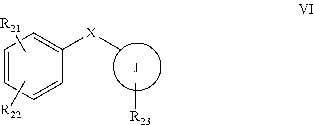

where:
X is O; J is 1-phenyl; R₂₁ is 2-NHSO₂CH₃; R₂₂ is 4-NO₂; and there is no R₂₃ group, (nimesulide); and
X is O; J is 1-oxo-inden-5-yl; R₂₁ is 2-F; R₂₂ is 4-F; and R₂₃ is 6-NHSO₂CH₃, (flosulide); and
X is O; J is cyclohexyl; R₂₁ is 2-NHSO₂CH₃; R₂₂ is 5-NO₂; and there is no R₂₃ group, (NS-398); and
X is S; J is 1-oxo-inden-5-yl; R₂₁ is 2-F; R₂₂ is 4-F; and R₂₃ is 6-N—SO₂CH₃. Na*, (L-745337); and
X is S; J is thiophen-2-yl; R²¹ is 4-F; there is no R²² group; and R²³ is 5-NHSO₂CH₃, (RWJ-63556); and
X is O; J is 2-oxo-5(R)-methyl-5-(2,2,2-trifluoroethyl)furan-(5H)-3-yl; R²¹ is 3-F; R²² is 4-F; and R²³ is 4-(p-SO₂CH₃)C₆H₄, (L-784512).

Further information on the applications of N-(2-cyclohexyloxynitrophenyl)methane sulfonamide (NS-398, CAS RN 123653-11-2), having a structure as shown in formula B-26, have been described by, for example, Yoshimi, N. et al., in Japanese J. Cancer Res., 90(4): 406-412 (1999); Falgueyret, J.-P. et al., in Science Spectra, available at: http://www.gbhap.com/Science_Spectra/20-1-article.htm (Jun. 6, 2001); and Iwata, K. et al., in Jpn. J. Pharmacol., 75(2): 191-194 (1997).

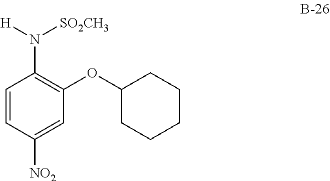

An evaluation of the antiinflammatory activity of the COX-2 inhibitor, RWJ 63556, in a canine model of inflammation, was described by Kirchner et al., in J Pharmacol Exp Ther 282, 1094-1101 (1997).

Other materials that can serve as the COX-2 inhibitor of the present invention include diarylmethylidenefuran derivatives that are described in U.S. Pat. No. 6,180,651. Such diarylmethylidenefuran derivatives have the general formula shown below in formula VII:

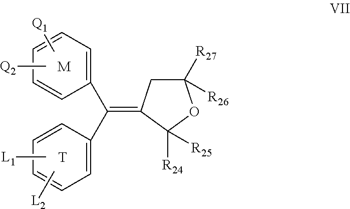

wherein:
the rings T and M independently are:
a phenyl radical,
a naphtyl radical,
a radical derived from a heterocycle comprising 5 to 6 members and possessing from 1 to 4 heteroatoms, or
a radical derived from a saturated hydrocarbon ring having from 3 to 7 carbon atoms;
   at least one of the substituents Q₁, Q₂, L₁ or L₂ is:
an —S(O)n-R group, in which n is an integer equal to 0, 1 or 2 and R is
a lower alkyl radical having 1 to 6 carbon atoms, or
a lower haloalkyl radical having 1 to 6 carbon atoms, or
an —SO₂NH₂ group;
and is located in the para position,
   the others independently being:
a hydrogen atom,
a halogen atom,
a lower alkyl radical having 1 to 6 carbon atoms,
a trifluoromethyl radical, or
a lower O-alkyl radical having 1 to 6 carbon atoms, or
   Q₁ and Q₂ or L₁ and L₂ are a methylenedioxy group; and
   R₂₄, R₂₅, R₂₆ and R₂₇ independently are:
a hydrogen atom,
a halogen atom,
a lower alkyl radical having 1 to 6 carbon atoms,
a lower haloalkyl radical having 1 to 6 carbon atoms, or
an aromatic radical selected from the group consisting of phenyl,
   naphthyl, thienyl, furyl and pyridyl; or,
   R₂₄, R₂₅ or R₂₆, R₂₇ are an oxygen atom, or
   R₂₄, R₂₅ or R₂₆, R₂₇, together with the carbon atom to which they are attached, form a saturated hydrocarbon ring having from 3 to 7 carbon atoms;
   or an isomer or prodrug thereof.

Particular materials that are included in this family of compounds, and which can serve as the COX-2 inhibitor in the present invention, include N-(2-cyclohexyloxynitrophenyl)methane sulfonamide, and (E)-4-[(4-methylphenyl) (tetrahydro-2-oxo-3-furanylidene)methyl]benzenesulfonamide.

COX-2 inhibitors that are useful in the present invention include darbufelone (Pfizer), CS-502 (Sankyo), LAS 34475 (Almirall Profesfarma), LAS 34555 (Almirall Profesfarma), S-33516 (Servier, see Current Drugs Headline News, at http://www.current-drugs.com/NEWS/Inflam1.htm, Oct. 4, 2001), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

COX-2 inhibitors that are useful in the invention can include the compounds that are described in U.S. Pat. Nos. 6,310,079; 6,306,890 and 6,303,628 (bicycliccarbonyl indoles); U.S. Pat. No. 6,300,363 (indole compounds); U.S. Pat. Nos. 6,297,282 and 6,004,948 (substituted derivatives of benzosulphonamides); U.S. Pat. Nos. 6,239,173, 6,169,188, 6,133,292; 6,020,343; 6,071,954; 5,981,576 ((methylsulfonyl)phenyl furanones); U.S. Pat. No. 6,083,969 (diarylcycloalkano and cycloalkeno pyrazoles); U.S. Pat. No. 6,222,048 (diaryl-2-(5H)-furanones; U.S. Pat. No. 6,077,869 (aryl phenylhydrazines); U.S. Pat. Nos. 6,071,936 and 6,001,843 (substituted pyridines); U.S. Pat. No. 6,307,047 (pyridazinone compounds); U.S. Pat. No. 6,140,515 (3-aryl-4-aryloxyfuran-5-ones); U.S. Pat. Nos. 6,204,387 and 6, 127,545 (diaryl pyridines); U.S. Pat. No. 6,057,319 (3,4-diaryl-2-hydroxy-2,5-dihydrofurans; U.S. Pat. No. 6,046,236 (carbocyclic sulfonamides); and U.S. Pat. Nos. 6,002,014; 5,994,381; and 5,945,539 (oxazole derivatives).

Preferred COX-2 inhibitors for the use according to the present invention include celecoxib (Celebrex®), rofecoxib (Vioxx®)), meloxicam, piroxicam, deracoxib, parecoxib, valdecoxib, etoricoxib, cimicoxib, a chromene derivative, a chroman derivative, N-(2-cyclohexyloxynitrophenyl)methane sulfonamide, COX189, ABT963, JTE-522, pharmaceutical acceptable salts, prodrugs or mixtures thereof. More preferred COX-2 inhibitors are celecoxib, parecoxib, valdecoxib, etoricoxib and rofecoxib.

According to a preferred embodiment, celecoxib (Celebrex®) or a pharmaceutical acceptable salt thereof is used. The term pharmaceutical acceptable salt includes salts that can be prepared according to known methods by those skilled in the art from the corresponding compound of the present invention, e.g. conventional metallic ion salts and organic salts.

Celecoxib can be administered at a dose of 50-1600 mg per day, preferably 200 to 600 mg, most preferably 400 mg per day. The administration can be carried out once or several times a day, preferably twice. The amount of celecoxib can be adapted depending on age, body weight and/or possible other diseases of the patient. Preferably, celecoxib is used in the form of tablets (Celebrex®) for oral administration.

Without intending to establish a certain theory as explanation for the observed effect of COX-2 inhibitors, the following mechanisms of action are taken into consideration.

There is no doubt that activation of COX-2 mediates inflammatory processes and that COX-2 is expressed in brain tissue. COX-2 can be activated by cytokines like IL-2, IL-6 and IL-10, and cytokine-activated COX-2 expression mediates further inflammatory processes. It was reported that IL-2 and soluble IL-2 receptors (Licino et al: Elevated levels of Interleukin-2 in neuroleptic-free schizophrenics. Am J Psychiatry 1993; 150: 1408-1410) (McAllister et al: Increases in CSF levels of Interleukin-2 in schizophrenia: effects of recurrence of psychosis and medication status. Am J Psychiatry 1995; 152: 1291-1297), soluble IL-6 receptors as a functional part of the IL-6 system (Müller et al: Soluble IL-6 Receptors in the serum and cerebrospinal fluid of paranoid schizophrenic patients. Eur Psychiatry 1997; 12: 294-299) and IL-10 (Van Kammen et al: Relationship between immune and behavioral measures in schizophrenia. In: G. Wieselmann (ed.) Current Update in Psychoimmunology, Springer Verlag 1997; Wien, N.Y., pp. 51-55) are increased in the cerebrospinal fluid of schizophrenic patients—the increase of the cytokines in the CNS may be accompanied by increased COX-2 expression. The effectiveness of COX-2 inhibitors, such as celecoxib, in the treatment of schizophrenia, might be based on the finding that celecoxib down-regulates the cytokine-induced CNS COX-2 activation.

Moreover, COX-2 inhibition seems to regulate the expression of adhesion molecules (Schwarz et al: Blood-CSF-Barrier impairment as indicator for an immune process in schizophrenia. Neurosci Letters 1998; 253: 201-203). Since adhesion molecule regulation is impaired in schizophrenia, leading to dysbalance and lack of communication between the peripheral and the CNS immune system, the effects of COX-2 inhibitors, such as celecoxib, in the treatment of schizophrenia, may also be related to the adhesion molecules ICAM-1 and VCAM-1, expecially regarding the negative symptoms (Schwarz et al: Levels of soluble adhesion molecules in schizophrenia: Relation to psychopathology. In: N. Müller (Hrg) Psychiatry, Psychoneuroimmunology, and Viruses. Springer Verlag Wien, 1999, NY, pp. 121-130; Müller N, Ackenheil M: Immunoglobulin and albumin contents of cerebrospinal fluid in schizophrenic patients: The relationship to negative sympomatology. Schizophrenia Res 1995; 14: 223-228).

According to a further embodiment of the present invention, a COX-2 inhibitor is used in combination with a neuroleptic drug or an antidepressant for the manufacture of a medicament for the treatment of psychiatric disorders such as schizophrenia, delusional disorders, affective disorders, autism or tic disorders. Combinations can also include a mixture of one or more COX-2 inhibitors with one or more neuroleptic agents or antidepressants. In particular, the combination of a COX-2 inhibitor with a neuroleptic drug is useful for the treatment of schizophrenia, whereas the combination of a COX-2 inhibitor with an antidepressant is applicable for the treatment of depressive disorders.

Both classical and atypical neuroleptics can be used for the add-on use according to the invention, atypical neuroleptics being preferred.

Examples of neuroleptic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benzisothiazolyl-piperazines; dibenzoxazepines, such as loxapine; dihydroindolones, such as molindone; aripiprazole; and derivatives thereof that have antipsychotic activity.

Examples of neuroleptic drugs that are preferred for use in the present invention are shown in Table 3.

TABLE 3
Neuroleptic drugs

| | | | | Dosage | |
|---|---|---|---|---|---|
| | | Route of | | Range and | |
| Common Name | Trade Name | Administration | Form | (Median)^{a} | |
| Clozapine | CLOZARIL | oral | tablets | 12.5-900 | mg/day |
| | | | | (300-900 | mg/day) |
| Olanzapine | ZYPREXA | oral | tablets | 5-25 | mg/day |
| | | | | (10-25 | mg/day) |
| Ziprasidone | GEODON | oral | capsules | 20-80 | mg/twice a day |
| | | | | (80-160 | mg/day) |
| Risperidone | RISPERDAL | oral solution | | 2-16 | mg/day |
| | | tablets | | (4-12 | mg/day) |
| Quetiapine | SEROQUEL | oral | tablets | 50-900 | mg/day |
| fumarate | | | | (300-900 | mg/day) |
| Sertindole | SERLECT | | | (4-24 | mg/day) |
| Amisulpride | | | | | |
| Haloperidol | HALDOL | oral | tablets | 1-100 | mg/day |
| | | | | (1-15 | mg/day) |
| Haloperidol | HALDOL | parenteral | injection | | |
| Decanoate | Decanoate | | | | |
| Haloperidol | HALDOL | oral | solution | | |
| lactate | INTENSOL | parenteral | injection | | |
| Chlorpromazine | THORAZINE | rectal | suppositories | 30-800 | mg/day |
| | | oral | capsules | (200-500 | mg/day) |
| | | | solution | | |
| | | | tablets | | |
| | | parenteral | injection | | |
| Fluphenazine | PROLIXIN | | | 0.5-40 | mg/day |
| | | | | (1-5 | mg/day) |
| Fluphenazine | PROLIXIN | parenteral | injection | (about one-half the | |
| decanoate | Decanoate | | | dosage shown for oral) | |
| Fluphenazine | PROLIXIN | parenteral | injection | (same as above) | |
| enanthate | | | | | |
| Fluphenazine | PROLIXIN | oral | elixer | | |
| hydrochloride | | | solution | | |
| | | | tablets | | |
| | | parenteral | injection | | |
| Thiothixene | NAVANE | oral | capsules | 6-60 | mg/day |
| | | | | (8-30 | mg/day) |
| Thiothixene | NAVANE | oral | solution | | |
| hydrochloride | | parenteral | injection | | |
| Trifluoperazine | STELAZINE | | | (2-40 | mg/day) |
| Perphenazine | TRILAFON | oral | solution | 12-64 | mg/day tablets |
| | | | tablets | (16-64 | mg/day) |
| | | parenteral | injection | | |
| Perpehazine and | ETRAFON | oral | tablets | | |
| Amitriptyline | TRIAVIL | | | | |
| hydrochloride | | | | | |
| Thioridazine | MELLARIL | oral | suspension | 150-800 | mg/day |
| | | | solution | (100-300 | mg/day) |
| | | | tablets | | |
| Mesoridazine | | | | (30-400 | mg/day) |
| Molindone | MOBAN | | | 50-225 | mg/day |
| | | | | (15-150 | mg/day) |
| Molindone | MOBAN | oral | solution | | |
| hydrochloride | | | | | |
| Loxapine | LOXITANE | | | 20-250 | mg/day |
| | | | | (60-100 | mg/day) |
| Loxapine | LOXITANE | oral | solution | | |
| hydrochloride | | parenteral | injection | | |
| Loxapine | LOXITANE | oral | capsules | | |
| succinate | | | | | |
| Pimozide | | | | (1-10 | mg/day) |

| | |
|---|---|
| Flupenthixol | |
| Promazine | SPARINE |
| Triflupromazine | VESPRIN |
| Chlorprothixene | TARACTAN |
| Droperidol | INAPSINE |
| Acetophenazine | TINDAL |
| Prochlorperazine | COMPAZINE |
| Methotrimeprazine | NOZINAN |
| Pipotiazine | PIPOTRIL |
| Ziprasidone | |
| Hoperidone | |
| Zuclopenthixol | |

Examples of tradenames and suppliers of selected neuroleptic drugs are as follows: clozapine (available under the tradename CLOZARIL®, from Mylan, Zenith Goldline, UDL, Novartis); olanzapine (available under the tradename ZYPREXA®, from Lilly; ziprasidone (available under the tradename GEODON®, from Pfizer); risperidone (available under the tradename RISPERDAL®, from Janssen); quetiapine fumarate (available under the tradename SEROQUEL®, Pharmaceutical Partners, Pasadena); thiothixene (available under the tradename NAVANE®, from Pfizer); trifluoperazine (10-[3-(4-methyl-1-piperazinyl)propyl]-2-(trifluoromethyl)phenothiazine dihydrochloride, available under the tradename STELAZINE®, from SmithKlein Beckman); perphenazine (available under the tradename TRILAFON®, from Schering); thioridazine (available under the tradename MELLARIL®, from Novartis, Roxane, Hi-Tech, Teva, and Alpharma); molindone (available under the tradename MOBAN®, from Endo); and loxapine (available under the tradename LOXITANE® from Watson). Furthermore, benperidol (Glianimon®), perazine (Taxilan®) or melperone (Eunerpan®) may be used.

Other preferred neuroleptic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®), prochlorperazine (available under the tradename COMPAZINE®), methotrimeprazine (available under the tradename NOZINAN®), pipotiazine (available under the tradename PIPOTRIL®), ziprasidone, and hoperidone.

Preferred neuroleptic drugs include risperidone and aripiprazole (from Bristol Myers Squibb Company, see e.g. Stahl S M; Dopamine-system stabilizers, aripiprazole and the next generation of antipsychotics, part 1, “goldilocks”-actions at dopamine receptors; J. Clin. Psychiatry 2001, 62, 11: 841-842).

The most preferred neuroleptic drug within the present invention is risperidone (Risperdal®), its manufacture and pharmacological activity is described in EP 0 196 132. Risperidone acts as an antagonist to neurotransmitters, in particular dopamine, and is used for the treatment of psychoses.

Within the present invention, the neuroleptic risperidone can be administered at a dose of 2-6 mg/day, preferably 4-5 mg. The dose for celecoxib may range from 50-1600 mg, preferably 200-600, more preferably 400 mg. Preferably, the administration occurs twice daily (in the morning and in the evening).

Various types of antidepressants can be used for the add-on use according to the present invention. Examples of antidepressants that are useful in the present invention include, but are not limited to: tricyclic antidepressants such as amitriptyline (5-(3-dimethylamino propylidene)-10,11-dihydro-5H-dibenzo[a,d]cyclohepten), amitriptyline oxide, desipramine (10,11-dihydro-5-(3-methylamino propyl)-5H-dibenz[b,f]azepin), dibenzepin (10-(2-dimethylamino ethyl)-5,11-dihydro-5-methyl-11H-dibenzo[b,e][1,4]diazepin-11-on), dosulepin (3-(6H-dibenzo[b,e]thiepin-11-yliden)-N,N-dimethylpropyl amine), doxepin (3-(6H-dibenz[b,e]oxepin-11-yliden)-dimethylpropyl amine), chloroimipramine, imipramine (5-(3-dimethylamino propyl)-5,11-dihydro-5H-dibenz[b,f]azepin), nortriptyline (3-(10,11-dihydro-5H-dibenzo[a, d]cyclohepten-5-yliden)-N-methyl-1-propane amine), mianserin (1, 2, 3, 4, 10,14b-hexahydro-2-methyl-dibenzo[c,f]pyrazino[1,2-a]azepin), maprotiline (N-methyl-9,10-ethanoanthracene-9 (10H)-propane amine), trimipramine (5-[3-dimethylamino)-2-methylpropyl]-10,11-dihydro-5H-dibenz[b,f]azepin) or viloxazine (RS)-2-(2-ethyoxy phenoxy methyl)-morpholine), modern antidepressants such as trazodone (2-{3-[4-(3-chlorophenyl)-1-piperazinyl]-propyl}-1,2,4-triazol[4,3-a]pyridine-3(2H)-on, nefazodone (2-{3-[4-(3-chlorophenyl)-1-piperazinyl]propyl}-5-ethyl-2,4-dihydro-4-(2-phenoxyethyl)-3H-1,2,4-triazol-3-on), mirtazapine ((±)-1,2,3,4,10,14b-hexahydro-2-methylpyrazino[2,1-a][2,3-c][2]benzazepin), venlafaxine ((±)-1-2-(dimethylamino)-1-(4-methoxyphenyl)-ethyl]cyclohexanol) or reboxetine ((±)-(2RS)-2-[(aSR)-a-(2-ethoxyphenoxy)benzyl]morpholine), inhibitors of monoaminooxidases such as tranylcypromine (trans-2-phenyl cyclopropyl amine), brofaromine or moclobemide (4-chloro-N-(2-morpholinoethyl)-benzamide), selective inhibitors of serotonin-uptake such as citalopram, paroxetine, fluoxetine ((RS)—N-methyl-3-phenyl-3-[4-(trifluoromethyl)phenoxy]propyl amine, available under the tradename PROZAC®)), fluvoxamine ((E)-5-methyoxy-4-(trifluoromethyl)-valerophenon-O-(2-aminoethyl)oxime) or sertraline ((1S-cis)-(+)-4-(3,4-dichlorophenyl)-1,2,3,4-tetrahydro-N-methyl-1-naphthalinamine), and vegetable antidepressants such as Hypericum (St. John's wort).

The invention is also directed to a novel kit-of-parts that is suitable for use in the treatment of psychiatric disorders such as schizophrenia, delusional disorders, affective disorders, autism or tic disorders, comprising a first dosage form comprising a neuroleptic agent or an antidepressant and a second dosage form comprising a COX-2 inhibitor or prodrug thereof, for simultaneous, separate or sequential administration.

According to a preferred embodiment, the dosage form comprising a neuroleptic agent or an antidepressant and the second dosage form comprising a COX-2 inhibitor are administered simultaneously.

The subject pharmaceutical kit-of-parts may be administered enterally (orally) or parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. Preferably the administration of a pharmaceutical kit comprising a COX-2 inhibitor and a neuroleptic or antidepressant occurs enterally (orally), in form of tablets.

The treatment of psychiatric disorders with COX-2 inhibitors, alone or in combination with a neuroleptic or antidepressant, may occur in addition to further drug therapies. Thus, tranquilizers may be used for the treatment of agitation, anxiety or sleep disturbances. Preferably lorazepam is used, which belongs to the class of benzodiazepines.

In the following, the invention will be discussed in more detail with reference to a patient study. Other embodiments within the scope of the claims herein will be apparent to one skilled in the art from consideration of the specification or practice of the invention as disclosed herein. The results of the patient study are graphically represented in the attached figures, which will be discussed in more detail in the following.

### DESCRIPTION OF THE FIGURES

FIG. 1 shows the comparison of the PANSS score during treatment with risperidone-celecoxib or risperidone-placebo.

FIG. 2 shows the comparison of the PANSS negative score during treatment with risperidone-celecoxib or risperidone-placebo.

FIG. 3 shows the comparison of the PANSS global score during treatment with risperidone-celecoxib or risperidone-placebo.

FIG. 4 shows the plasma levels of risperidone and 9-OH-risperidone during treatment with risperidone-celecoxib or risperidone-placebo.

FIG. 5 shows the biperiden and benzodiazepine use during treatment with risperidone-celecoxib or risperidone-placebo.

FIG. 6 shows the effect of oral administration of rofecoxib on the behavior of mice in the forced swim test. n=8 per group.

FIG. 7 shows the effect of oral administration of valdecoxib on the behavior of mice in the forced swim test. n=9 for the control group, n=9 for the group treated with 4 mg/day valdecoxib, n=10 for the group treated with 20 mg/day valdecoxib.

FIG. 8 shows the effect of oral administration of etoricoxib on the behavior of mice in the forced swim test. n=9 for the control group, n=10 for the group treated with 4 mg/day etoricoxib, n=9 for the group treated with 20 mg/day etoricoxib.

FIG. 9 shows the effect of oral administration of cimicoxib on the behavior of mice in the forced swim test. n=9 for the control group, n=10 for the group treated with 4 mg/day cimicoxib, n=9 for the group treated with 20 mg/day cimicoxib.

FIG. 10 shows a comparison of HamD scores during therapy with celecoxib or placebo (ANOVA, estimated marginal means; advantage of celecoxib-group: Greenhouse-Geisser-corrected F=3.220; df 2.434; p=0.035).

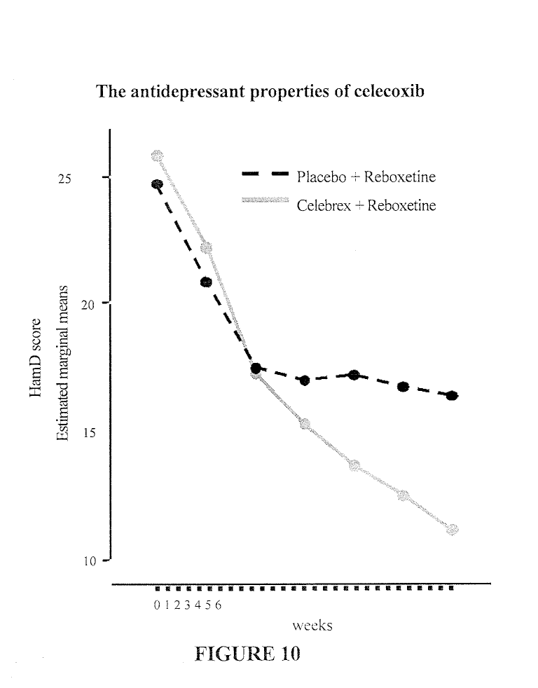

FIG. 11 shows a comparison of HamD-17 and MADRS scores during therapy with cimicoxib+sertraline or placebo+sertraline

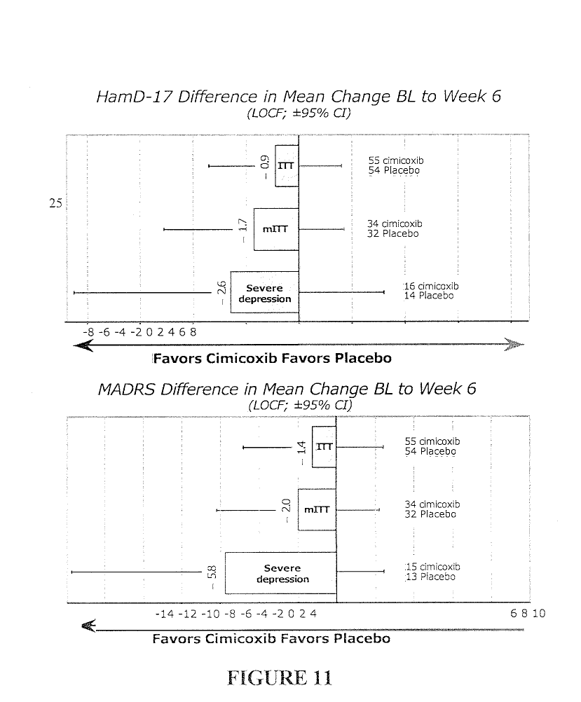

FIG. 12 shows the remission rates according to HamD-17 and MADRS scores during therapy with cimicoxib+sertraline or placebo+sertraline. Remission is defined as a HamD-17 score of less than 7 or a MADRS score of equal or less than 10.

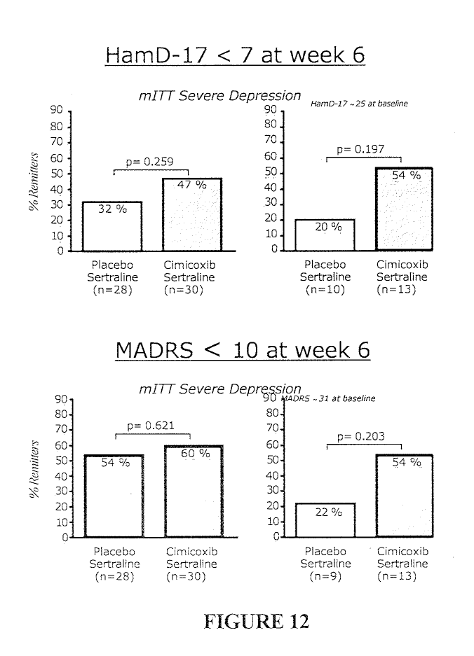

### EXAMPLES

### Example 1

The study was performed as a single-center, double-blind, placebo-controlled, randomized, parallel-groupe valuation of the combination therapy with celecoxib and risperidone versus a monotherapy with risperidone and placebo in schizophrenic patients. The study included 50 patients fulfilling the criteria for the diagnosis of schizophrenia according to DSM IV (American Psychiatric Association (1994), Diagnostic and Statistical Manual of Mental Disorders, 1st Edition, American Psychiatric Press, Washington D.C.), of whom 25 belonged to the risperidone-placebo and 25 to the risperidone-celecoxib group. No significant differences were present between the two patient groups were found with regard to age, sex, duration or severity of the disease or psychopathology, risperidone dose or risperidone-plasma levels.

The patients received 2-6 mg/day of risperidone (Risperdal®), and depending on to which group they belonged, 400 mg/day of celecoxib (2×200 mg Celebrex® mornings and evenings) or placebo over 5 weeks after a brief wash-out period of earlier antipsychotic medication. During the wash-out period, a benzodiazepine preparation (mostly lorazepam) was prescribed, if necessary. Patients with agitation, anxiety, or sleeping problems were also medicated with lorazepam during the study.

The psychopathology of the patients was assessed using the positive and negative syndrome scale (PANSS) (Kay et al., Schizophr. Bull. 1987, 13: 261-276). The extrapyramidal side effects were assessed by the EPS scale (Simpson and Angus, Acta Psychiat. Scand. 1970 (Suppl.), 212). The use of biperiden was monitored as a possible indicator for side effects of the antipsychotic medication.

In order to exclude the chance that possible differences in the therapeutic effectiveness between the two groups might be due to non-compliance during the risperidone therapy or to differences in risperidone metabolism, the plasma levels of risperidone or 9-OH-risperidone were monitored during the study.

The statistics were performed according to the criterion of “last observation carried forward” (LOCF), i.e., the last PANSS scores of the patients who dropped out before the end of the study were carried forward to all subsequent observation days. For the comparison of the main efficacy parameter, the mean change in the PANSS between the two treatment groups, t-tests for independent samples were employed. With reference to the underlying hypothesis of a better outcome of the celecoxib-risperidone group, a significance of p<0.05 was calculated in the one-tailed t-test and used as the basis for the estimation of the sample size (statistical power) and for the comparison of the groups. For all other comparisons, two-tailed t-tests were used.

At the start of the study, in the risperidone-celecoxib group (average age 35.9±12.8 years), the PANSS total score was 71.8±17.1, the PANSS global score was 34.0±8.5, the PANSS positive score was 19.0±5.9 and the PANSS negative score was 18.7±6.3. In the risperidone-placebo group (average age 35.5±13.6 years), the PANSS total score was 75.4±12.9, the PANSS global score was 37.2±7.1, the PANSS positive score was 17.2±4.6 and the PANSS negative score was 21.1±5.5. Consequently, there was no significant difference in the PANSS total score or any of the subscales.

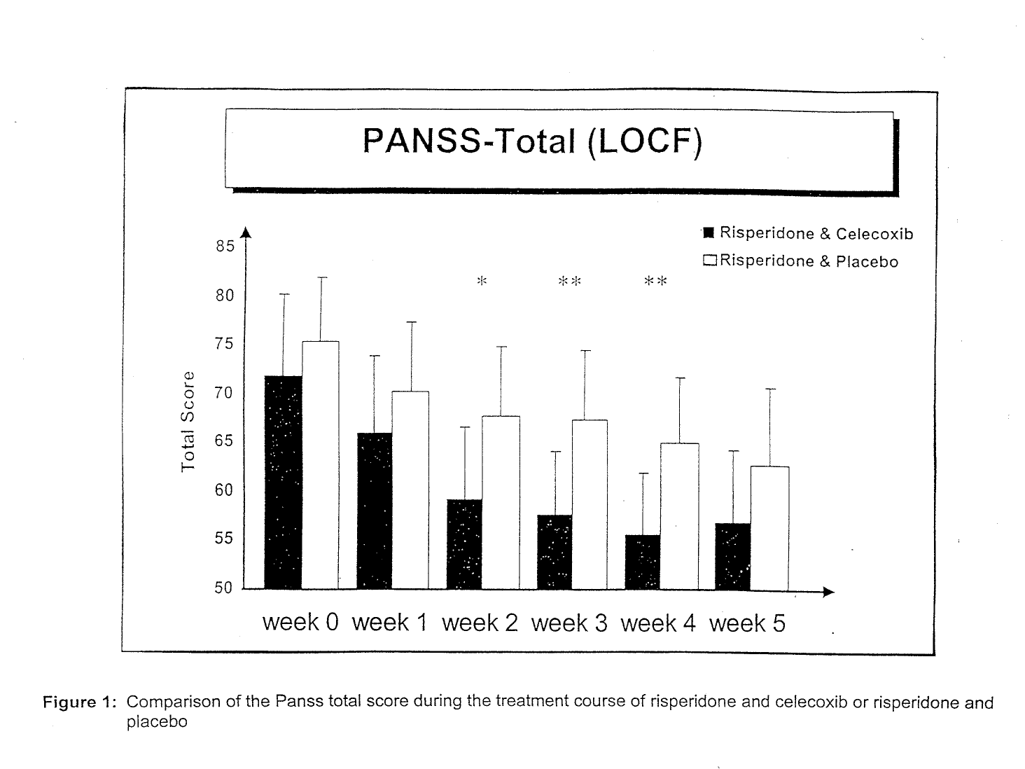

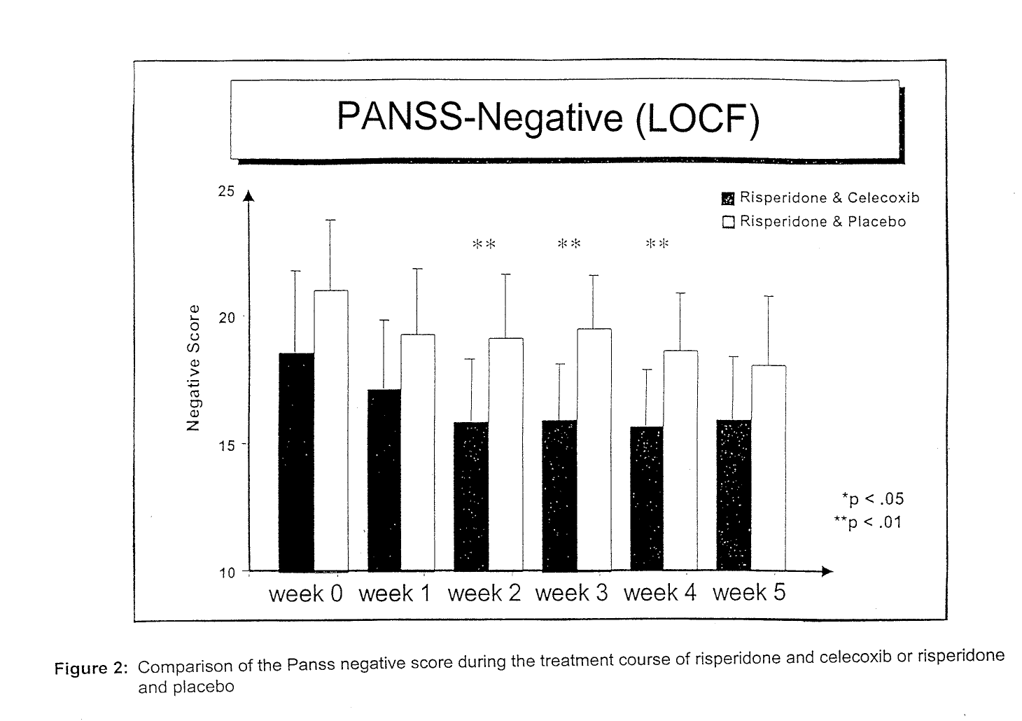

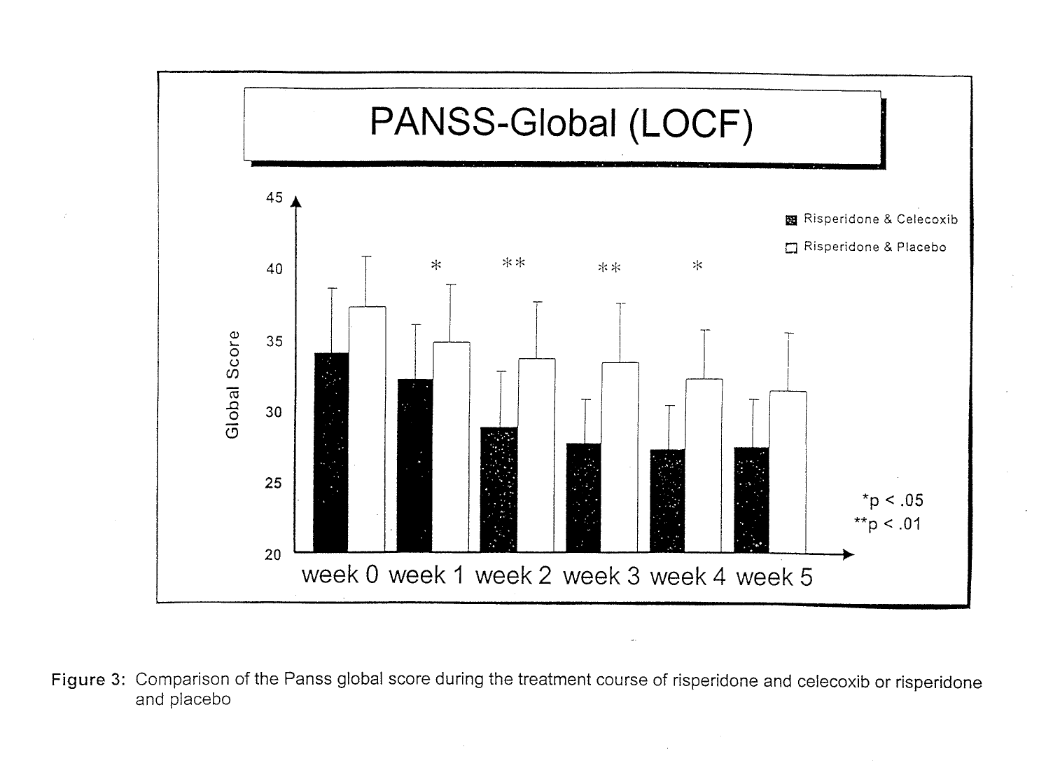

During the five-week therapy, a significant improvement of the PANSS total score and the subscales is observed in both groups of schizophrenic patients. The results of the PANSS total score are shown in FIG. 1, of the PANSS negative score in FIG. 2, of the PANSS global score in FIG. 3 and of the PANSS positive score in Table 4.

TABLE 4
Comparison of the PANSS positive score

| | celecoxib and | placebo and | | |
|---|---|---|---|---|
| time | risperidone | risperidone | t¹ | p² |
| week 0 | 19.0 ± 5.9 | 17.2 ± 4.6 | 1.22 | n.s.³ |
| week 1 | 16.7 ± 5.5 | 16.2 ± 4.6 | 0.36 | n.s. |
| week 2 | 14.4 ± 5.0 | 15 ± 4.5 | 0.42 | n.s. |
| week 3 | 14.0 ± 4.7 | 14.5 ± 4.6 | 0.36 | n.s. |
| week 4 | 12.8 ± 4.4 | 14.2 ± 4.4 | 1.16 | n.s. |
| week 5 | 13.4 ± 5.6 | 13.3 ± 4.4 | 0.11 | n.s. |

¹t represents the statistical random sample distribution
²p represents the statistical power (probability).
³n.s. means no statistical significance.

In the celecoxib-risperidone group, the two-tailed t-tests between the baseline and week 5 gave the following values: PANSS total score p<0.0001, PANSS global score p<0.0001, PANSS positive score p<0.0001, PANSS negative score p<0.001. In the placebo-risperidone group, the t-tests between the baseline and week 5 gave the following values: PANSS total score p<0.002, PANSS global score p<0.003, PANSS positive score p<0.002, PANSS negative score p<0.02.

The improved effectiveness of the combination therapy with celecoxib-risperidone in comparison to risperidone monotherapy is clearly shown by the significantly lower PANSS global scores after the 2, 3, 4 and 5 weeks of treatment (FIG. 3). With regard to the total and negative score, significantly lower scores were recorded after 2, 3 and 4 weeks in the celecoxib-risperidone group (FIGS. 1 and 2).

The mean daily dose of risperidone is shown in Table 5; no statistically significant difference was found between the two treatment groups.

TABLE 5
Mean risperidone dose mg/day

| | celecoxib and | placebo and | |
|---|---|---|---|
| time | risperidone | risperidone | difference |
| week 1 | 4.1 ± 0.6 | 4.0 ± 0.8 | n.s. |
| week 2 | 4.5 ± 0.6 | 4.4 ± 1.1 | n.s. |
| week 3 | 4.8 ± 0.8 | 4.9 ± 1.4 | n.s. |
| week 4 | 5.0 ± 1.0 | 4.9 ± 1.4 | n.s. |
| week 5 | 4.9 ± 1.0 | 5.1 ± 1.5 | n.s. |

¹ n.s. means no statistical significance.

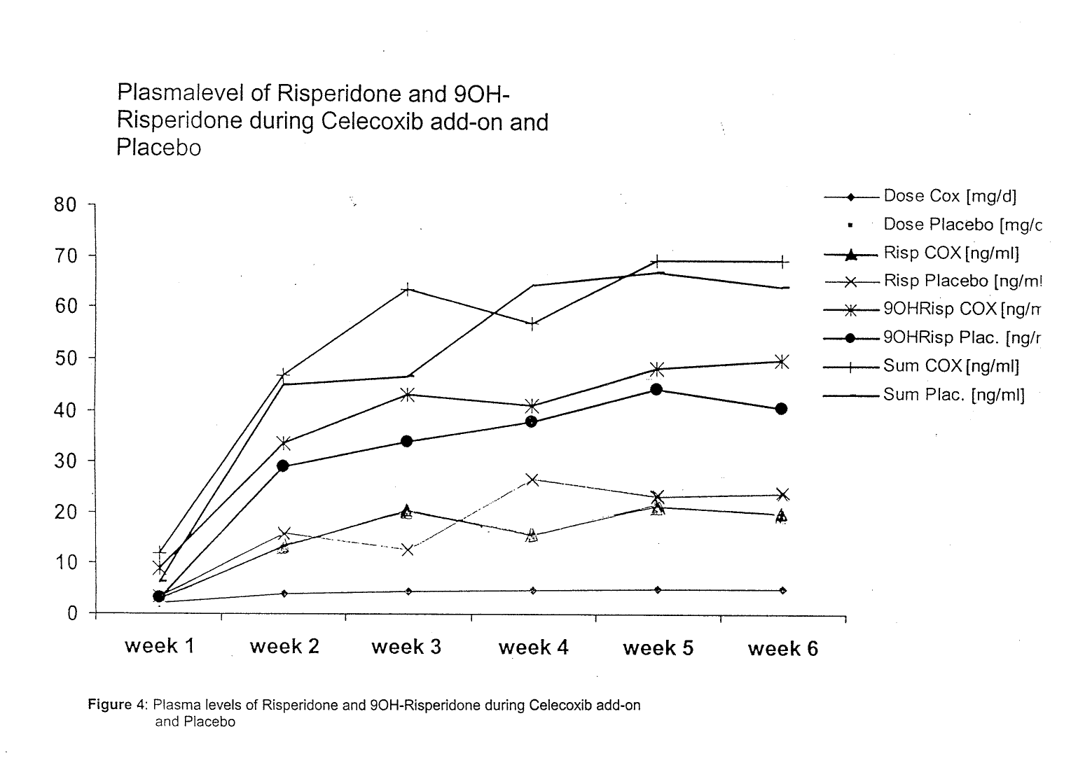

The differences in the plasma levels of risperidone or the metabolite 9-OH-risperidone shown in FIG. 4 were also without statistical significance (the present FIG. 4 differs from FIG. 4 of the German patent application priority document due to a calculation error in said priority document).

Therefore, it could be excluded that the observed differences in the therapeutic effectiveness between the two groups are due to incompatibility during the risperidone therapy or differences in risperidone metabolism. The therapeutic benefit of the combined therapy has to be attributed to the COX-2 inhibitor, celecoxib.

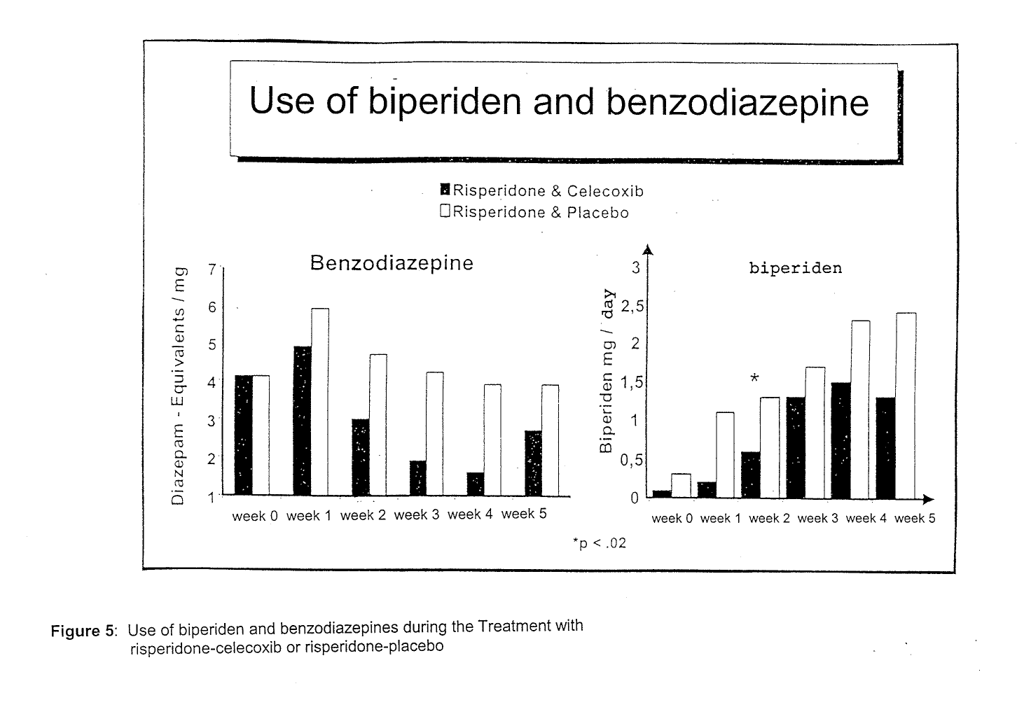

With respect to the extrapyramidal side effects, no statistically significant differences were found in the EPS scale. The use of biperiden is shown in FIG. 5 and was calculated as cumulative weekly dose. The values were lower in the celecoxib-risperidone group, and reached statistical significance at week 2 (p<0.02).

A detailed analysis of items of the PANSS-Scale which discriminate good celecoxib-responders from the placebo group revealed that therapeutic effects of celecoxib are especially found on the items “lack of contact” (item 3 of the negative subscale), “emotional isolation” (item 2 of the negative subscale),“passive-apathic isolation” (item 4 of the negative subscale),“social withdrawal” (item 16 of the general psychopathology subscale),“depression” (item 6 of the general psychopathology subscale) and “motor retardation” (item 6 of the general psychopathology subscale).

Furthermore, a factor analysis showed that especially items which can subsumed under the label “agitation” show a good therapeutic response to celecoxib, but not to placebo. All those items reflect psychopathological symptoms which are typically found in depressive states. Therefore this detailed analysis points to a therapeutic efficiency in depressive states.

Moreover, “passive-apathic isolation”, “motor retardation”, “social withdrawal”, or “lack of contact” are—often more severe expressed than in depressive states—also core-symptoms of childhood autism.

The combination of celecoxib and risperidone according to the present invention thus shows improved results compared to the monopreparation risperidone with regard to effectiveness in the treatment of schizophrenia. Furthermore, it was observed that the beneficial effects of the add-on therapy occurred faster in patients with a recent onset of the disorder and that the celecoxib therapy was useful in the treatment of depressive states.

### Example 2

### The Selective COX-2 Inhibitor Rofecoxib Possesses Antidepressant Activity

Various behavioral test paradigms have been developed for evaluating the antidepressant properties of novel drugs in animals. One of the most reliable and specific paradigm is the forced swim test which has been successfully used to determine the effectiveness of antidepressants, evaluate new pharmaceutical compounds and validate animal models of depression (Porsolt et al. (1977) Arch. Int. Pharmacodym. 229:327-336; Porsolt (2000) Rev. Neurosci. 11:53-58; Rénńric et al. (2002)Behav. Brain Res. 136:521-532; Page et al. (2003) Psychopharmacology 165:194-201; Kelliher et al. (2003) Psychoneuroendocrinology 28:332-347). The test consists of placing a mouse for a period of 5 minutes into a glass cylinder containing a water depth of at least 15 cm. Under such circumstances, a mouse cannot touch the bottom of the cylinder and is thus forced to swim. Time, latency and frequency of struggling/swimming versus floating are scored as behavioral parameters. Floating (i.e. movements made only for keeping balance and breath) can be interpreted as a depression-like behavior that reflects either a failure of persistent escape-directed behavior (i.e. behavioral despair) or the development of passive behavior that disengages the animal from active forms of coping with stress stimuli. By contrast, increased struggling (i.e. vigorous attempts to escape) and swimming indicates active coping behavior and can be interpreted as an opposite to depression-like behavior. Treatment with existing antidepressants has been shown to reduce the total time spent floating while increasing the time spent swimming and/or struggling, which is interpreted as an improvement in depression-like behavior (Rńnńric et al. (2002) Behav. Brain Res. 136:521-532; Page et al. (2003) Psychopharmacology 165:194-201; Kelliher et al. (2003) Psychoneuroendocrinology 28:332-347).

The antidepressant activity of rofecoxib was assessed in animals according to the forced swim test. Briefly, rofecoxib was given orally by gavage to mice either in a single acute administration of 10 mg or in a repetitive chronic administration of 4 mg per day (2 mg at 9:00 in the morning, 2 mg at 18:00 in the evening) for 28 days. Control animals received a negative control consisting of water only. The forced swim test was performed 4 hour after the last administration of rofecoxib. All experiments were performed using a selected DBA/2Ola mouse strain that displays characteristics of being anxious and responds to antidepressant treatment. All observed results were confirmed statistically using the one-way ANOVA test.

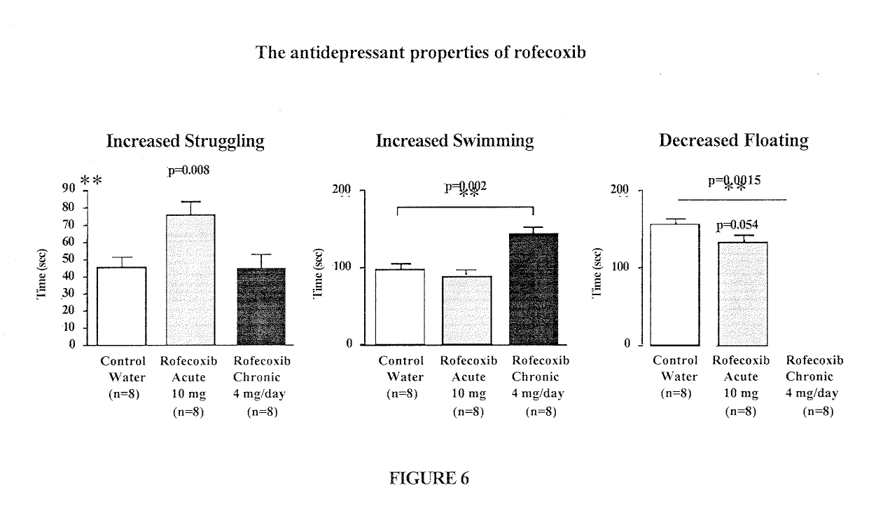

As shown in FIG. 6, acute and chronic administration of rofecoxib statistically increased either struggling or swimming times while decreasing floating time. These results demonstrate that rofecoxib increases stress coping behavior, which is interpreted as an improvement of depression-like behavior.

### Example 3

### The COX-2 Inhibitor Valdecoxib Possesses Antidepressant activity

The antidepressant activity of valdecoxib was assessed in DBA/2Ola mice according to the forced swim test. Briefly, chronic administration of valdecoxib was performed by oral gavage for 28 days at a concentration of 4 mg per day (2 mg at 9:00 in the morning, 2 mg at 18:00 in the evening) and 20 mg per day (10 mg at 9:00 in the morning, 10 mg at 18:00 in the evening). Control animals received a placebo consisting of water only. The behavior of individual animals was assessed using the forced swim test 24 hours after the last administration of valdecoxib. A pre-exposure of 5 minutes to the test was done 4 hours after the last administration of valdecoxib. All observed results were confirmed statistically using the one-way ANOVA test.

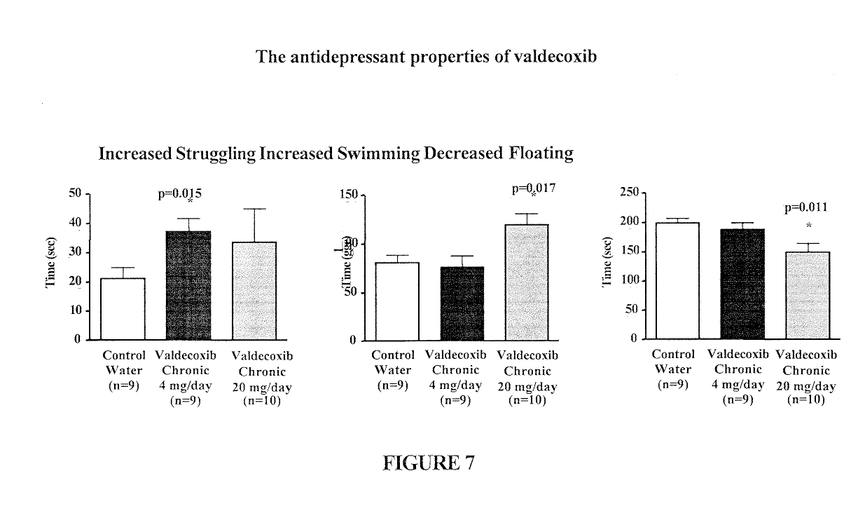

As shown in FIG. 7, chronic administration of valdecoxib statistically increased either struggling or swimming times while decreasing floating time. These results demonstrate that valdecoxib increases stress coping behavior, which is interpreted as an improvement of depression-like behavior.

### Example 4

### The COX-2 Inhibitor Etoricoxib Possesses Antidepressant Activity

The antidepressant activity of etoricoxib was assessed in DBA/201a mice according to the forced swim test. Briefly, etoricoxib was chronically administered to mice by oral gavage for 28 days at a concentration of 4 mg per day (2 mg at 9:00 in the morning, 2 mg at 18:00 in the evening) and 20 mg per day (10 mg at 9:00 in the morning, 10 mg at 18:00 in the evening). Control animals received a placebo consisting of water only. The behavior of individual animals was assessed using the forced swim test 24 hours after the last administration of etoricoxib. A pre-exposure of 5 minutes to the test was done 4 hours after the last administration of etoricoxib. All observed results were confirmed statistically using the one-way ANOVA test.

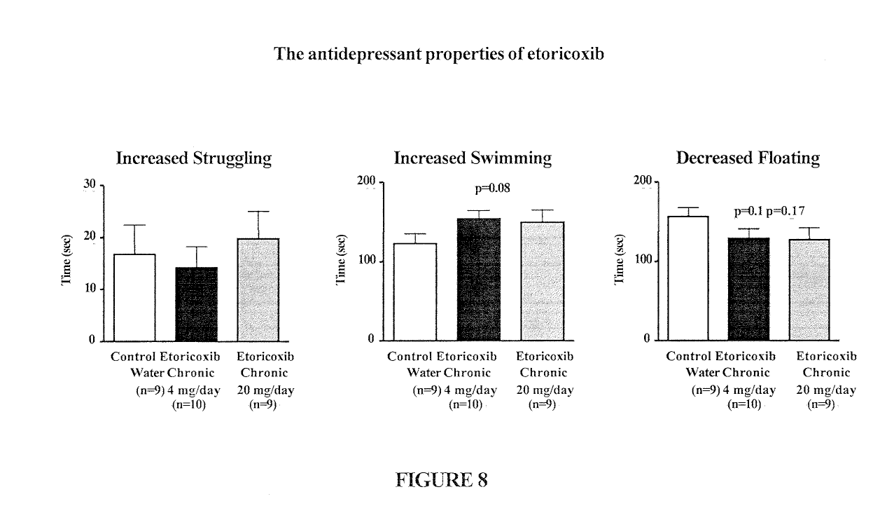

As shown in FIG. 8, chronic administration of etoricoxib statistically increased or showed a tendency to increase either struggling or swimming times while decreasing floating time. These results demonstrate that etoricoxib increases stress coping behavior, which is interpreted as an improvement of depression-like behavior.

### Example 5

### The COX-2 Inhibitor Cimicoxib Possesses Antidepressant Activity

The antidepressant activity of the COX-2 inhibitor Cimicoxib was assessed in DBA/2Ola mice using the forced swim test. Briefly, chronic administration of cimicoxib was performed by oral gavage to mice for 28 days at a concentration of 2 or 10 mg per kg bodyweight per day (1 or 5 mg at 9:00 in the morning, 1 or 5 mg at 18:00 in the evening). Control animals received a placebo consisting of water only. The behavior of individual animals was assessed using the forced swim test 4 hours after the last administration of cimicoxib. All observed results were confirmed statistically using the one-way ANOVA test.

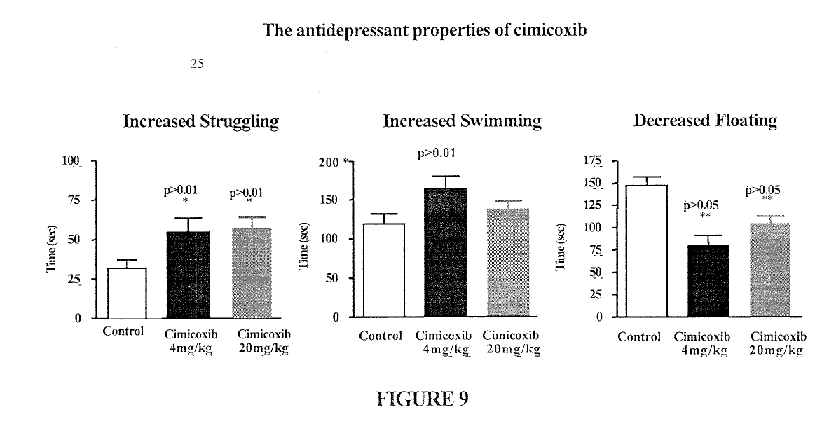

As shown in FIG. 9, chronic administration of cimicoxib resulted in a statistically significant increase of either struggling or swimming times and a decrease in floating time. These results demonstrate that cimicoxib increases stress coping behavior, which is interpreted as an improvement of depression-like behavior. This experiment yet further supports the use of COX-2 inhibitors in the treatment of psychiatric disorders, such as schizophrenia and depression.

### Example 6

### The COX-2 Inhibitor Celecoxib Possesses Antidepressant Activity in Human Depressed Patients

The antidepressant activity of the selective COX-2 inhibitor celecoxib was demonstrated in a clinical study involving 40 depressed patients.

The study was conducted as a double-blind, randomized, placebo controlled, prospective parallel group trial of celecoxib add-on to reboxetine. The treatment period lasted 42 days (6 weeks) after a wash-out period of at least three days in pre-medicated patients. All patients suffered from MD (DSM IV: 296.2× single depressive episode or 296.3× recurrent depressive episode). 40 patients (20 f, 20 m) aged between 23 and 63 years were included in the study. 37 of the patients included were in-patients. 12 males and 8 females were included in the celecoxib-group and 8 males and 12 females in the placebo-group. 34 patients were included in Munich and six patients in Munster. Patients suffering from psychotic depression were excluded. Each patient was included after written informed consent. The study was examined by the ethics committee of the medical faculty of the university of Munich.

The psychopathology of the patients was assessed by raters, who had undergone a training program, using the Hamilton-Depression scale, 17-item version (HamD), 24 Assessment of psychopathology and other examinations were performed at weekly intervals. At baseline, no difference could be seen between the groups regarding the severity of depression.

During the wash-out and the treatment periods the patients additionally received the benzodiazepine lorazepam for acute agitation or anxiety.

Celecoxib and placebo were administered in identical capsules produced by the pharmacy of the medical faculty Munich according to the randomization scheme. The dose of reboxetine was flexible and ranged from 4 mg/day to 10 mg/day, according to clinical needs. Celecoxib was administered at a dose of 400 mg/day. Reboxetine was started with 2 mg for two days before administering 4 mg, celecoxib was titrated from 200 mg/day to 400 mg/day within three days.

In order to exclude the chance that any differences in treatment response between the groups might be due to noncompliance during reboxetine therapy or to differences in reboxetine metabolism (e.g. through reboxetine—celecoxib interactions), reboxetine plasma levels were monitored during the study. An overview on the characteristics of the patients and doses of drugs is shown in Table 6.

TABLE 6
Overview on characteristics of patients receiving
celecoxib or placebo (mean ± SD)

| | celecoxib | | placebo | |
|---|---|---|---|---|
| | (n = 20) | | (n = 20) | |
| sex | 12 m, 8 f | | 8 m, 12 f | |
| age range | 25-63 | years | 23-65 | years |
| mean age | 44.5 ± 11.6 | years | 44.3 ± 13.5 | years |
| age of onset | 36.2 ± 12.4 | years | 37.5 ± 15.0 | years |
| episodes (including | | | | |
| present) | | | | |
| range | 1-11 | | 1-5 | |
| mean | 2.5 ± 2.3 | | 2.4 ± 1.2 | |
| hospitalizations | | | | |
| (incl. pres.) | | | | |
| range | 1-11 | | 0-5 | |
| mean | 1.1 ± 0.3 | | 1.6 ± 1.2 | |
| duration present episode | 17.0 ± 21.7 | | 18.7 ± 20.8 | |
| (weeks) | | | | |
| reboxetine dose | | | | |
| range | 4-10 | mg/day | 4-10 | mg/day |
| mean | 6.79 ± 0.82 | mg/day | 6.81 ± 1.14 | mg/day |
| benzodiazepine dose | | | | |
| (diazepam-equivalents) | | | | |
| range | 0-7 | mg/day | 0-10 | mg/day |
| mean | 2.4 mg ± 3.0 | mg/day | 2.7 mg ± 3.1 | mg/day |

At inclusion into the study the severity of depression ranged from 15 to 38 points on the HamD scale. The drop-out rate was relatively high in both groups. 10 patients dropped out from the celecoxib group before the trial end. Five of them were excluded or refused further treatment in the study due to a lack of therapeutic efficacy, four patients were excluded due to side-effects of the treatment (increase in blood-pressure, sleep-disturbance, difficulties in miction or erection, exanthema of the skin). Regarding the point of time for drop-out, patients from the celecoxib-group dropped out later: three patients after three weeks, five after four weeks and two during the last week of the trial. Of the latter two, one patient refused the last visit because he was discharged from the hospital and felt healthy.

From the placebo-group, twelve patients dropped before the end of the study. Nine of them were excluded or denied further treatment due to a lack of therapeutic efficacy, three patients were excluded due to side-effects of the treatment (nausea, agitation, sinus-tachychardia). Three patients dropped-out already after only two weeks, four after three weeks and five after four weeks.

In the celecoxib group, no cardiovascular events or side-effects were observed, neither clinically nor by ECG surveillance.

For statistics, analysis of variance was used for the HamD scale. The degrees of freedom for the within-subjects comparisons were corrected for deviance from sphericity (Greenhouse-Geisser). Post-hoc t tests were used for the weekly comparison of HamD scores. An intent to treat analysis was performed using the “last observation carried forward” (LOCF) procedure. For the comparison of reboxetine plasma levels, the pair-wise t test was used.

We claim:

1. A method for treating psychiatric disorders comprising administering a composition comprising a COX-2 inhibitor to a subject in need of such treatment, wherein the Cox-2 inhibitor is a COX-2 inhibitor of the following formula IV: 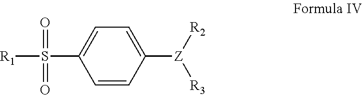
wherein:
Z is selected from the group consisting of partially unsaturated or unsaturated heterocyclyl and partially unsaturated or unsaturated carbocyclic rings;
R₁ is selected from the group consisting of methyl or amino:
R₂ is selected from the group consisting of heterocyclyl, cycloalkyl, cycloalkenyl and aryl, wherein R₂ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and
R₃ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;
or a prodrug thereof.

2. The method according to claim 1 wherein said psychiatric disorders comprise schizophrenia, delusional disorders, affective disorders, autism or tic disorders.

3. The method according to claim 1 wherein said psychiatric disorders comprise chronic schizophrenic psychoses, schizoaffective psychoses, temporary acute psychotic disorders, depressive episodes, recurring depressive episodes, manic episodes or bipolar affective disorders.

4. The method according to claim 1 wherein the COX-2 inhibitor is selected from the group consisting of celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963 or JTE-522, pharmaceutically acceptable salts, prodrugs and mixtures thereof.

5. The method according to claim 4 wherein the COX-2 inhibitor is celecoxib or a pharmaceutically acceptable salt thereof.

6. The method according to claim 5 wherein celecoxib or a pharmaceutically acceptable salt thereof is administered in an amount of 50-1600 mg per day, preferably 200 to 600 mg, most preferably 400 mg.

7. The method according to claim 1 wherein the composition is administered orally.

8. The method according to claim 1 wherein the composition comprises a neuroleptic drug or an antidepressant.

9. The method according to claim 8 wherein said psychiatric disorders comprise schizophrenia, delusional disorders, affective disorders, autism or tic disorders.

10. The method according to claim 8 wherein said psychiatric disorders comprise chronic schizophrenic psychoses, schizoaffective psychoses, temporary acute psychotic disorders, depressive episodes, recurring depressive episodes, manic episodes and bipolar affective disorders.

11. The method according to claim 8 wherein the COX-2 inhibitor is selected from the group consisting of celecoxib, rofecoxib, meloxicam, piroxicam, deracoxib, parecoxib, valdecoxib, etoricoxib, a chromene derivative, a chroman derivative, N-(2-cyclohexyloxynitrophenyl)methane sulfonamide, COX189, ABT963 or JTE-522, pharmaceutically acceptable salts, prodrugs and mixtures thereof.

12. The method according to claim 8 wherein the COX-2 inhibitor is celecoxib or a pharmaceutically acceptable salt thereof.

13. The method according to claim 8 wherein the neuroleptic drug is selected from the group consisting of clozapine, olanzapine, ziprasidone, risperidone, aripiprazole, quetiapine, quetiapine fumarate, sertindole, amisulpride, haloperidol, haloperidol decanoate, haloperidol lactate, chlorpromazine, fluphenazine, fluphenazine decanoate, fluphenazine enanthate, fluphenazine hydrochloride, thiothixene, thiothixene hydrochloride, trifluoperazine, perphenazine, amitriptyline, thioridazine, mesoridazine, molindone, molindone hydrochloride, loxapine, loxapine hydrochloride, loxapine succinate, pimozide, flupenthixol, promazine, triflupromazine, chlorprothixene, droperidol, actophenazine, prochlorperazine, methotrimeprazine, pipotiazine, ziprasidone, hoperidone, zuclopenthixol, and mixtures thereof.

14. The method according to claim 8 wherein the antidepressant is selected from the group consisting of amitriptyline, amitriptyline oxide, desipramine, dibenzepin, dosulepin, doxepin, chloroimipramine, imipramine, nortriptyline, mianserin, maprotiline, trimipramine, viloxazine, trazodone, nefazodone, mirtazapine, venlafaxine, reboxetine, tranylcypromine, brofaromine, moclobemide, citalopram, paroxetine, fluoxetine, fluvoxamine, sertraline, Hypericum (St. John's Wort), and mixtures thereof.

15. The method according to claim 12 wherein the neuroleptic drug is risperidone or aripiprazole.

16. The method according to claim 15 wherein celecoxib or a pharmaceutically acceptable salt thereof and risperidone are administered in an amount of (1) 50-1600 mg, preferably 200-600 mg, and 2-6 mg, respectively or (2) 400 mg and 4-5 mg, respectively.

17. The method according to claim 8 wherein the composition is administered orally.

18. The method according to claim 1 wherein a tranquilizer, preferably lorazepam, is also administered.

19. A kit suitable for use in the treatment of affective disorders, the kit comprising a first dosage form comprising a neuroleptic drug or an antidepressant and a second dosage form comprising a COX-2 inhibitor or prodrug thereof, for simultaneous, separate or sequential administration, wherein the Cox-2 inhibitor is a COX-2 inhibitor of the following formula IV: 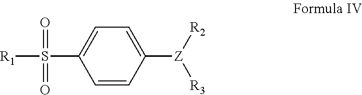
wherein:
Z is selected from the group consisting of partially unsaturated or unsaturated heterocyclyl and partially unsaturated or unsaturated carbocyclic rings;
R₁ is selected from the group consisting of methyl or amino:
R₂ is selected from the group consisting of heterocyclyl, cycloalkyl, cycloalkenyl and aryl, wherein R₂ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and
R₃ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;
or a prodrug thereof.

20. The kit according to claim 19, wherein the neuroleptic is selected from the group consisting of clozapine, olanzapine, ziprasidone, risperidone, quetiapine, quietiapine fumarate, sertindole, amisulpride, haloperidol, haloperidol decanoate, haloperidol lactate, chlorpromazine, fluphenazine, fluphenazine decanoate, fluphenazine enanthate, fluphenazine hydrochloride, thiothixene, thiothixene hydrochloride, trifluoperazine, perphenazine, amitriptyline, thioridazine, mesoridazine, molindone, molindone hydrochloride, loxapine, loxapine hydrochloride, loxapine succinate, pimozide, flupenthixol, promazine, triflupromazine, chlorprothixene, droperidol, actophenazine, prochlorperazine, methotrimeprazine, pipotiazine, ziprasidone, hoperidone, zuclopenthixol, and mixtures thereof.

21. The kit according to claim 19, wherein the antidepressant is selected from amitriptyline, amitriptyline oxide, desipramine, dibenzepin, dosulepin, doxepin, chloroimipramine, imipramine, nortriptyline, mianserin, maprotiline, trimipramine, viloxazine, trazodone, nefazodone, mirtazapine, venlafaxine, reboxetine, tranylcypromine, brofaromine, moclobemide, citalopram, paroxetine, fluoxetine, fluvoxamine, sertraline, Hypericum (St. John's Wort), and mixtures thereof.

22. The kit according to claim 19 wherein the COX-2 inhibitor is selected from the group consisting of celecoxib, rofecoxib, meloxicam, piroxicam, deracoxib, paracoxib, valdecoxib, etroicoxib, a chromene derivative, a chroman derivative, N-(2-cyclohexyloxynitrophenyl)methane sulfonamide, COX189, ABT963 or JTE-522, pharmaceutically acceptable salts, prodrugs and mixtures thereof.

23. The kit according to claim 19, wherein the COX-2 inhibitor celecoxib or a pharmaceutically acceptable salt thereof as COX-2 inhibitor and risperidone as neuroleptic drug.

24. The kit according to claim 23, wherein celecoxib or a pharmaceutically acceptable salt thereof and risperidone are in an amount of 50-1600 mg and 2-6 mg, respectively.

25. The method according to anyone of claim 8 wherein a tranquilizer, preferably lorazepam, is administered additionally

26. The method according to claim 1 wherein the COX-2 inhibitor is cimicoxib.

27. The method according to claim 8 wherein the COX-2 inhibitor is cimicoxib.

28. The kit according to claim 19 wherein the COX-2 inhibitor is cimicoxib.
